SEQUENCE LISTING (4 sequences)

SEQ ID NO: 1 - DNA, 384 bp, Artificial Sequence
  immunoglobulin light chain F
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatttcgcagtgtattactgtcatcagagtagtcgtttacctcacactttcggccaagggaccaaggtggagatcaaacgtaca

SEQ ID NO: 2 - protein, 128 aa, Artificial Sequence
  immunoglobulin light chain F
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 3 - DNA, 411 bp, Artificial Sequence
  immunoglobulin heavy chain A
atggagtttgggctgagctgggttttccttgttgctatattaaaaggtgtccagtgtgaggttcagctggtgcagtctgggggaggcttggtaaagcctggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 4 - protein, 137 aa, Artificial Sequence
  immunoglobulin heavy chain A
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

(19) United States
(12) Patent Application Publication
(10) Pub. No.: US 2006/0205810 A1
(43) Pub. Date: Sep. 14, 2006

(54) Platinum therapeutic combinations

(75) Inventors: Chen Zong, Metuchen, NJ (US); Paul Kirschmeier, Basking Ridge, NJ (US); Paul T. Medeiros, Easton, PA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/284,016

(22) Filed: Nov. 21, 2005

Related U.S. Application Data
(60) Provisional application No. 60/630,581, filed on Nov. 24, 2004.

(51) Int. Cl. A61K 31/28

(52) U.S. Cl. 514/492

(57) ABSTRACT

The present invention provides combination compositions comprising Pt based compounds, including satraplatin, along with another chemotherapeutic agent such as temozolomide or lonafarnib. The combinations are useful for the prevention or treatment of cancer. Method of using the combinations to treat or prevent cancer are also provided

This application claims the benefit of U.S. provisional patent application no. 60/630,581; filed Nov. 24, 2004 which is herein incorporated by reference in its entirety.

### FIELD OF THE INVENTION

The field of the present invention relates to methods and compositions for treating or preventing cancer.

### BACKGROUND OF THE INVENTION

Satraplatin (JM216; structure shown below) is an orally administered platinum drug that is currently undergoing clinical trials. It is rapidly metabolized to several different metabolites in the blood (Carr et al., Cancer Chemother. Pharmacol. 50:9-15 (2002). DNA damage inflicted by satraplatin is repaired in vitro with similar kinetics to those of cisplatin and oxaliplatin by the mammalian nucleotide excision repair pathway.

Satraplatin has been used in the treatment of cancer in humans. For example, clinical trials have demonstrated that satraplatin is effective against small cell lung cancer (SCLC). The tumor response rate was 10 of 26 (38%) with no complete responses and a median overall survival time of 210 days (Fokkema et al., J. Clin. Oncol. 17: 3822-3827 (1999)). Satraplatin (JM-216) plus prednisone has been used in treating patients with hormone refractory prostate cancer. Satraplatin in combination with paclitaxel was used in the treatment of advanced malignancies (Hainsworth et al., Invest. New Drugs 20(1):55-61 (2002)). Satraplatin has also been administered along with anti-cancer radiation therapy (George et al., Invest. New Drugs 19(4):303-310.(2001); Amorino et al., Cancer Chemother. Pharmacol. 46(5):423-426 (2000)), with uracil/ftorafur (UFT) (DeMario et al., Cancer Chemother. Pharmacol. 1999;43(5):385-388 (1999)), with UFT and leucorovin (Oncology (Huntingt);11(9 Suppl 10):26-9 (1997)), with docetaxel and with etoposide (Rose et al., Cancer Chemother. Pharmacol. 40(1):51-56 (1997)).

Cell culture studies have shown that satraplatin as well as its metabolite JM118 (see infra) can partially circumvent intrinsic and acquired resistance to cisplatin suggesting a Pt-resistance mechanism based on tolerance or increased repair, rather than decreased initial Pt-DNA adduct formation (Fokkema et al. Biochem. Pharmacol. 63:1989-1996 (2002)). Satraplatin undergoes extensive biotransformation in vivo and very little intact parent drug remains in the systemic circulation following oral administration. In vitro studies where satraplatin was incubated with fresh human whole blood have shown a half-life for the disappearance of the drug of only 6.3 min and its conversion mainly to JM118 and platinated serum albumin whereas 62% of the added platinum was associated with red blood cells (Carr et al., Cancer Chemother. Pharmacol. 50:9-15 (2002)). JM118 is an active metabolite of satraplatin and has been shown to form intrastrand cross-links and to have a greater cytotoxicity than cisplatin with respect to human ovarian carcinoma cells. JM118, binds to DNA similarly to cisplatin, forming intra- and interstrand cross-links between adjacent purine bases such as two isomeric 1,2-d(GpG) intrastrand cross-links. The X-ray crystal structure of the major adduct between this molecule and a DNA dodecamer at 2.4 Å resolution showed details of the distortion of the DNA duplex including a global bend angle of about 38° and a dihedral angle between platinated guanine bases of approximately 31° in a very similar manner to that of cisplatin and oxaliplatin. Therefore, differences in activity between these drugs may result from gross conformational distortions in DNA structure following platinum intrastrand cross-link formation (Silverman et al., J. Biol. Chem. 277: 49743-49749 (2002)).

Although satraplatin has been shown to be an effective, orally bioavailable anti-cancer treatment, there exists a need in the art for chemotherapeutic methods and compositions exhibiting enhanced anti-cancer activity.

### SUMMARY OF THE INVENTION

The scope of the present invention provides compositions comprising
(a) any Pt based compound set forth herein, specifically, under the “Platinum based Compounds” section of the instant specification; in association with
(b) any “other chemotherapeutic agent” set forth herein, specifically, under the “Other Chemotherapeutic Agents” section of the instant specification.

The present invention comprises a composition or kit comprising (a) a Pt based compound represented by the structural formula: 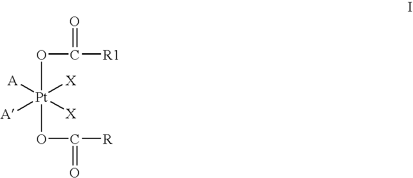
wherein A and A′ are NH₃ or a C1-C10 cyclic, straight-chain or branched-chain alkyl amine; R and R1 are selected from the group consisting of hydrogen, C1-C10 alkyl, alkenyl, aryl, aralkyl, alkylamino and alkoxy; and X is selected from the group consisting of halogen, alkyl mono-carboxylate and alkyl di-carboxylate or a Pt based compound represented by the structural formula 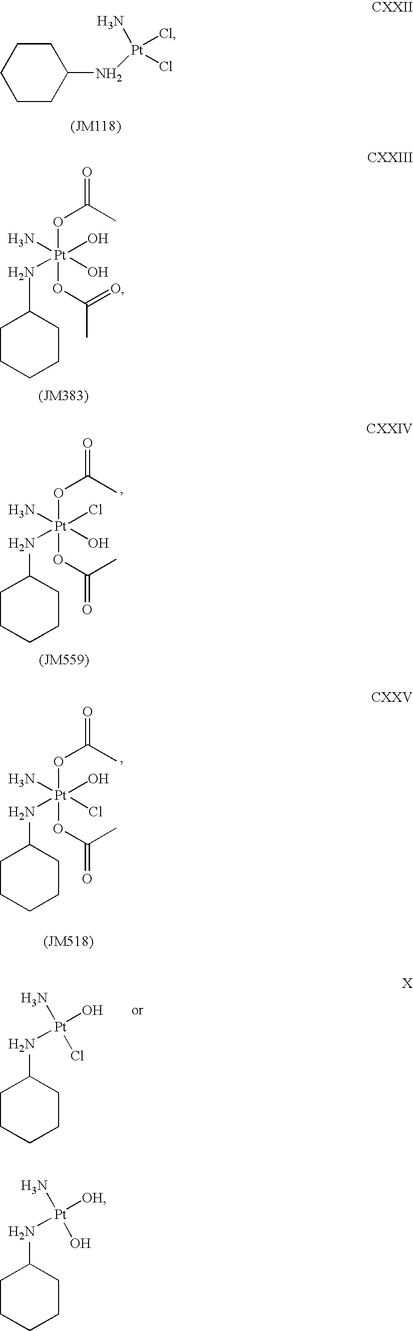
in association with (b) another chemotherapeutic agent represented by the following structural formula: 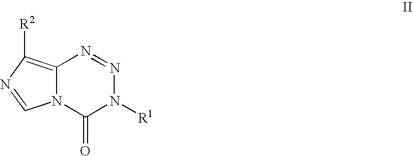
wherein R¹ represents a hydrogen atom, or a straight- or branched-chain alkyl, alkenyl or alkynyl group containing up to 6 carbon atoms, each such group being unsubstituted or substituted by from one to three substituents selected from halogen atoms, straight- or branched-chain alkoxy, alkylthio, alkylsuphinyl and alkylsulphonyl groups containing up to 4 carbon atoms, and optionally substituted phenyl groups, or wherein R¹ represents a cycloalkyl group; and R² represents a carbamoyl group optionally carries, on the nitrogen atom, one or two groups selected from straight- and branched-chain alkyl and alkenyl groups, containing up to 4 carbon atoms, and cycloalkyl groups. In an embodiment of the invention the Pt based compound is 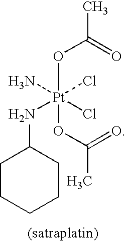
An embodiment of the invention comprises a pharmaceutical composition comprising said composition and a pharmaceutically acceptable carrier. In embodiment of the invention, satraplatin is formulated into an oral dosage form (e.g., a pill, a caplet, a tablet and a capsule).

The present invention also provides a composition or kit comprising (a) a Pt based compound represented by the structural formula: 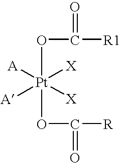
wherein A and A′ are NH₃ or a C1-C10 cyclic, straight-chain or branched-chain alkyl amine; R and R1 are selected from the group consisting of hydrogen, C1-C10 alkyl, alkenyl, aryl, aralkyl, alkylamino and alkoxy; and X is selected from the group consisting of halogen, alkyl mono-carboxylate and alkyl di-carboxylate or a Pt based compound represented by the structural formula 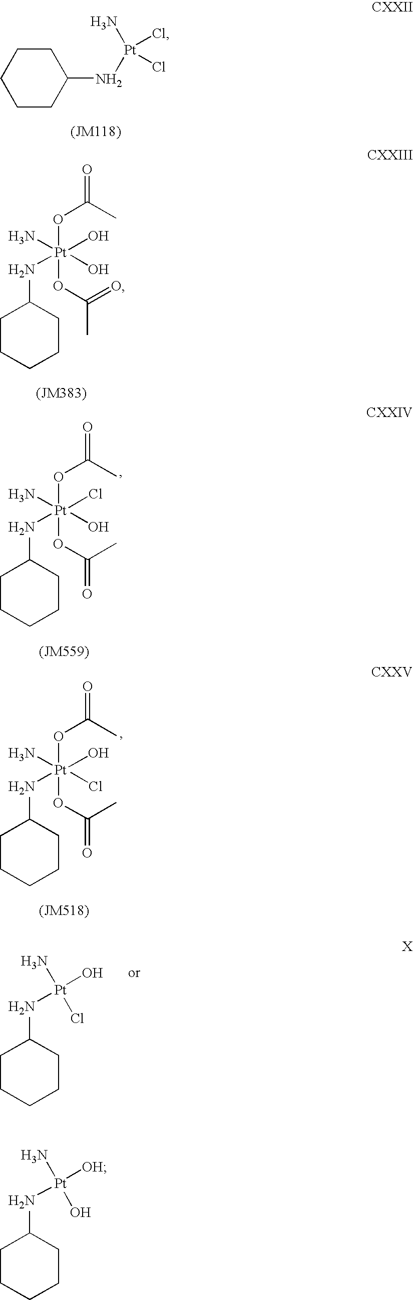
in association with
(b) another chemotherapeutic agent represented by the following structural formula: 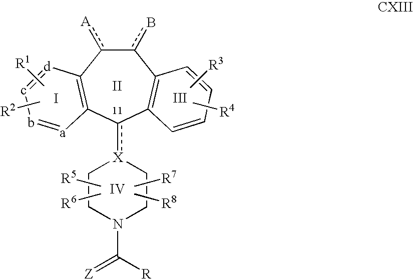
   or a pharmaceutically acceptable salt or solvate thereof, wherein: one of a, b, c and d represents N or NR⁹ wherein R⁹ is O—, —CH₃ or —(CH₂)ₙCO₂H wherein n is 1 to 3, and the remaining a, b, c and d groups represent CR¹ or CR²; or each of a, b, c, and d are independently selected from CR¹ or CR²; each R¹ and each R² is independently selected from H, halo, —CF₃, —OR¹⁰, —COR¹⁰, —SR¹⁰, —S(O)ₜR¹¹ (wherein t is 0, 1 or 2), —SCN, —N(R¹⁰)₂, —NO₂, —OC(O)R¹⁰, —CO₂R¹⁰, —OCO₂R¹¹, —CN, —NHC(O)R¹⁰, —NHSO₂R¹⁰, —CONHR¹⁰, —CONHCH₂CH₂OH, —NR¹⁰COOR¹¹, —SR¹¹ C(O)OR¹¹, 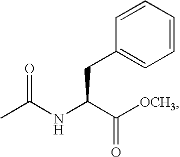
   —SR¹¹N(R⁷⁵)₂ (wherein each R⁷⁵ is independently selected from H and —C(O)OR¹¹), benzotriazol-1-yloxy, tetrazol-5-ylthio, or substituted tetrazol-5-ylthio, alkynyl, alkenyl or alkyl, said alkyl or alkenyl group optionally being substituted with halo, —OR¹⁰ or —CO₂R¹⁰; R³ and R⁴ are the same or different and each independently represents H, any of the substituents of R¹ and R², or R³ and R⁴ taken together represent a saturated or unsaturated C₅-C₇ fused ring to the benzene ring; R⁵, R⁶, R⁷ and R⁸ each independently represents H, —CF₃, —COR¹⁰, alkyl or aryl, said alkyl or aryl optionally being substituted with —OR¹⁰, —SR¹⁰, —S(O)ₜR¹¹, —NR¹⁰COOR¹¹, —N(R¹⁰)₂, —NO₂, —COR¹⁰, —OCOR¹⁰, —OCO₂R¹¹, —CO₂R¹⁰, OPO₃R¹⁰ or one of R⁵, R⁶, R⁷ and R⁸ can be taken in combination with R⁴⁰ as defined below to represent —(CH₂)ᵣ— wherein r is 1 to 4 which can be substituted with lower alkyl, lower alkoxy, —CF₃ or aryl, or R⁵ is combined with R⁶ to represent ═O or ═S and/or R⁷ is combined with R⁸ to represent ═O or ═S; R¹⁰ represents H, alkyl, aryl, or aralkyl; R¹¹ represents alkyl or aryl; X represents N, CH or C, which C may contain an optional double bond, represented by the dotted line, to carbon atom 11; the dotted line between carbon atoms 5 and 6 represents an optional double bond, such that when a double bond is present, A and B independently represent —R¹⁰, halo, —OR¹¹, —OCO₂R¹¹ or —OC(O)R¹⁰, and when no double bond is present between carbon atoms 5 and 6, A and B each independently represent H₂, —(OR¹¹ )₂; H and halo, dihalo, alkyl and H, (alkyl)₂, —H and —OC(O)R¹⁰, H and —OR¹⁰, ═O, aryl and H, ═NOR¹⁰ or —O—(CH₂)ₚ—O— wherein p is 2, 3 or 4; R represents R⁴⁰, R⁴², R⁴⁴, or R⁵⁴, as defined below; R⁴⁰ represents H, aryl, alkyl, cycloalkyl, alkenyl, alkynyl or -D wherein -D represents 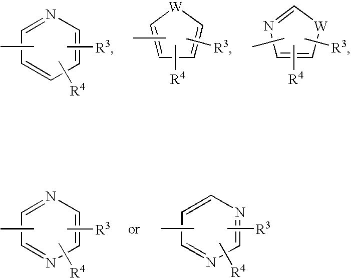
   wherein R³ and R⁴ are as previously defined and W is O, S or NR¹⁰ wherein R¹⁰ is as defined above; said R⁴⁰ cycloalkyl, alkenyl and alkynyl groups being optionally substituted with from 1-3 groups selected from halo, —CON(R¹⁰)₂, aryl, —CO₂R¹⁰, —OR¹², —SR¹², —N(R¹⁰)₂, —N(R¹⁰)CO₂R¹¹, —COR¹², —NO₂ or D, wherein -D, R¹⁰ and R¹¹ are as defined above and R¹² represents R¹⁰, —(CH₂)ₘOR¹⁰ or —(CH₂)_{q}CO₂R¹⁰ wherein R¹⁰ is as previously defined, m is 1 to 4 and q is 0 to 4; said alkenyl and alkynyl R⁴⁰ groups not containing —OH, —SH or —N(R¹⁰)₂ on a carbon containing a double or triple bond respectively; or R⁴⁰ represents phenyl substituted with a group selected from —SO₂NH₂, —NHSO₂CH₃, —SO₂NHCH₃, —SO₂CH₃, —SOCH₃, —SCH₃, or —NHSO₂CF₃, preferably, said group is located in the para position of the phenyl ring; or R⁴⁰ represents a group selected from

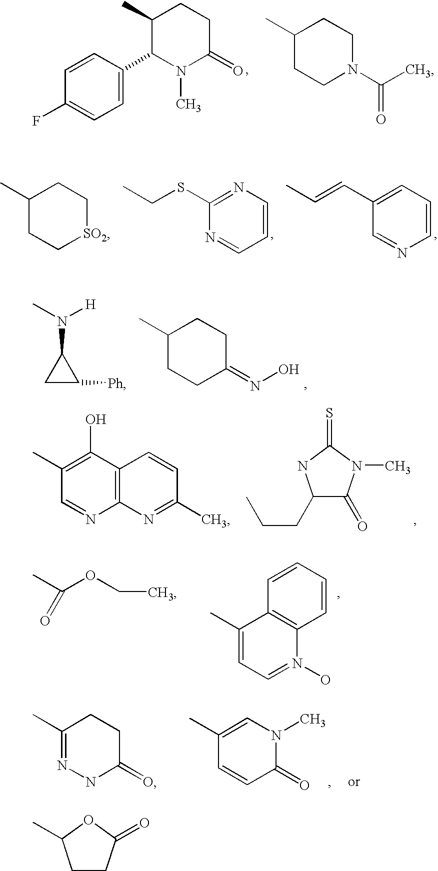

R⁴² represents 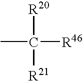
wherein R²⁰, R²¹ and R⁴⁶ are each independently selected from the group consisting of:

(1) H;

(2) —(CH₂)_{q}SC(O)CH₃ wherein q is 1 to 3;

(3) —(CH₂)_{q}OSO₂CH₃ wherein q is 1 to 3;

(4) —OH;

(5) —CS(CH₂)_{w}(substituted phenyl) wherein w is 1 to 3 and the substitutents on said substituted phenyl group are the same substitutents as described below for said substituted phenyl;

(6) —NH₂;

(7) —NHCBZ;

(8) —NHC(O)OR²² wherein R²² is an alkyl group having from 1 to 5 carbon atoms, or R²² represents phenyl substituted with 1 to 3 alkyl groups;

(9) alkyl;

(b 10) —(CH₂)ₖphenyl wherein k is 1 to 6;

(11) phenyl;

(12) substituted phenyl wherein the substituents are selected from the group consisting of: halo, NO₂, —OH, —OCH₃, —NH₂, —NHR²², —N(R²²)₂, alkyl, —O(CH₂)ₜphenyl (wherein t is from 1 to 3), and —O(CH₂)ₜsubstituted phenyl (wherein t is from 1 to 3);

(13) naphthyl;

(14) substituted naphthyl, wherein the substituents are as defined for substituted phenyl above;

(15) bridged polycyclic hydrocarbons having from 5 to 10 carbon atoms;

(16) cycloalkyl having from 5 to 7 carbon atoms;

(17) heteroaryl;

(18) hydroxyalkyl;

(19) substituted pyridyl or substituted pyridyl N-oxide wherein the substituents are selected from methylpyridyl, morpholinyl, imidazolyl, 1-piperidinyl, 1-(4-methylpiperazinyl), —S(O)ₜR¹¹, or any of the substituents given above for said substituted phenyl, and said substitutents are bound to a ring carbon by replacement of the hydrogen bound to said carbon;

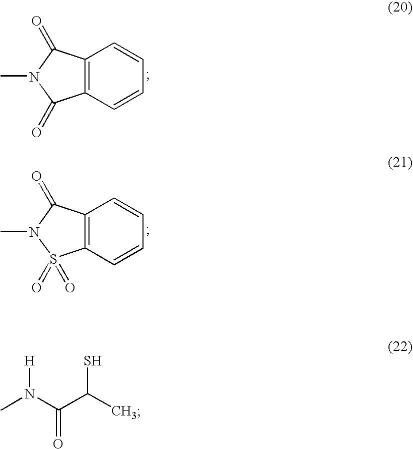

(23) —NHC(O)—(CH₂)ₖ-phenyl or —NH(O)—(CH₂)ₖ-substitued phenyl, wherein said k is as defined above;

(24) piperidine Ring V: 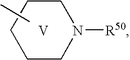
wherein R⁵⁰ represents H, alkyl, alkylcarbonyl, alkyloxycarbonyl, haloalkyl, or —C(O)NH(R¹⁰) wherein R¹⁰ is H or alkyl;; Ring V includes 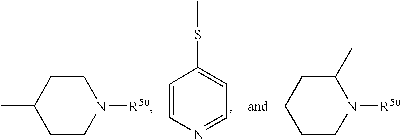
examples of Ring V include:

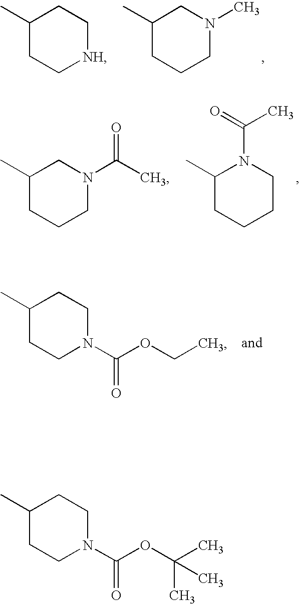

(25) —NHC(O)CH₂C₆H₅ or —NHC(O)CH₂-substituted-C₆H₅;

(26) —NHC(O)OC₆H₅;

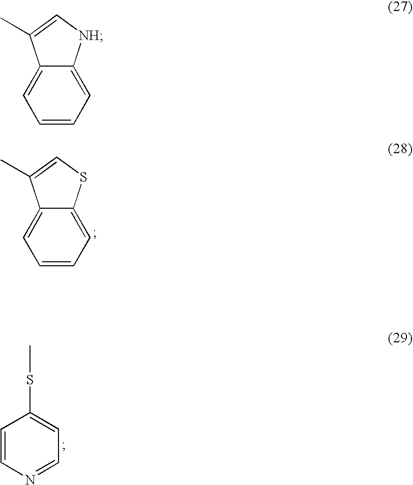

(30) —OC(O)-heteroaryl, for example

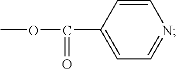

(31) —O-alkyl (e.g., —OCH₃); and

(32) —CF₃;

(33) —CN;

(34) a heterocycloalkyl group of the formula

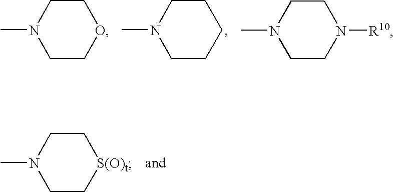

(35) a piperidinyl group of the formula 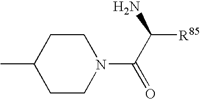
wherein R⁸⁵ is H, alkyl, or alkyl substituted by —OH or —SCH₃; or R²⁰ and R²¹ taken together form a ═O group and the remaining R⁴⁶ is as defined above; or

Two of R²⁰, R²¹ and R⁴⁶ taken together form piperidine Ring V 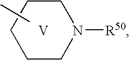
wherein R50 represents H, alkyl (e.g., methyl), alkylcarbonyl (e.g., CH3C(O)—), alkyloxycarbonyl (e.g., —C(O)O-t-C4H9, —C(O)OC2H5, and —C(O)OCH₃), haloalkyl (e.g., trifluro-methyl), or —C(O)NH(R10) wherein R10 is H or alkyl; Ring V includes 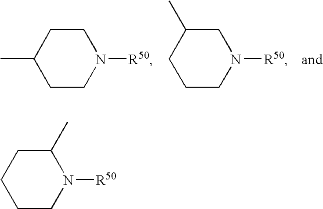
examples of Ring V include: 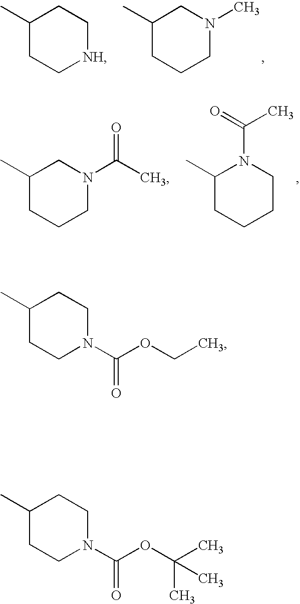
with the proviso R46, R20, and R21 are selected such that the carbon atom to which they are bound does not contain more than one heteroatom (i.e., R46, R20, and R21 are selected such that the carbon atom to which they are bound contains 0 or 1 heteroatom);R⁴⁴ represents 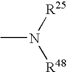
wherein R²⁵ represents heteroaryl, N-methylpiperdinyl or aryl; and R⁴⁸ represents H or alkyl; R⁵⁴ represents an N-oxide heterocyclic group of the formula (i), (ii), (iii) or (iv): 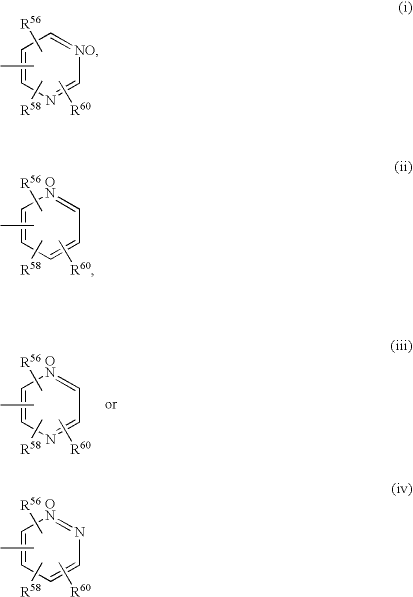
wherein R⁵⁶, R⁵⁸, and R⁶⁰ are the same or different and each is independently selected from H, halo, —CF₃, —OR¹⁰, —C(O)R¹⁰, —SR¹⁰,
—S(O)ₑR¹¹ (wherein e is 1 or 2), —N(R¹⁰)₂, —NO₂, —CO₂R¹⁰, ——OCO₂R¹¹, —OCOR¹⁰, alkyl, aryl, alkenyl or alkynyl, which alkyl may be substituted with —OR¹⁰, —SR¹⁰ or —N(R¹⁰)₂ and which alkenyl may be substituted with OR¹¹ or SR¹¹; or R⁵⁴ represents an N-oxide heterocyclic group of the formula (ia), (iia), (iiia) or (iva): 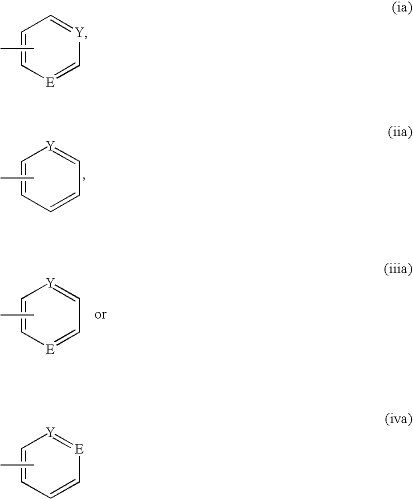
   wherein Y represents N⁺—O⁻ and E represents N; or

R⁵⁴ represents an alkyl group substituted with one of said N-oxide heterocyclic groups (i), (ii), (iii), (iv), (ia), (iia), (iiia) or (iva); Z represents O or S such that R can be taken in combination with R⁵, R⁶, R⁷ or R⁸ as defined above, or R represents R⁴⁰, R⁴², R⁴⁴ or R⁵⁴. Examples of R20, R21, and R46 for the above formulas include:

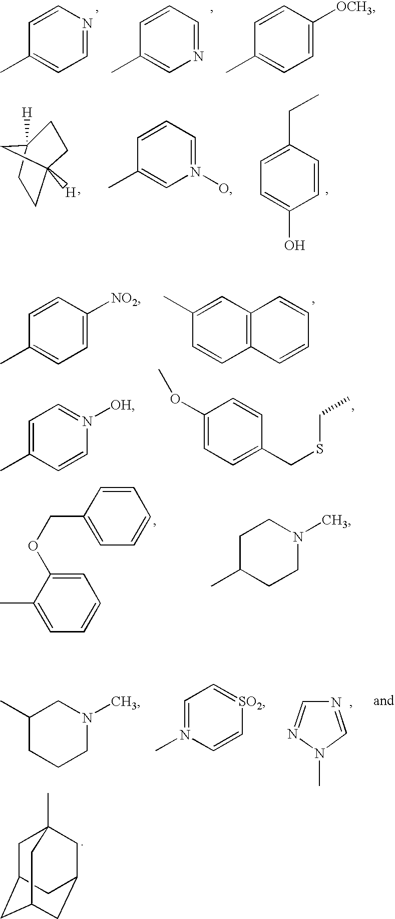

Examples of R25 groups include: 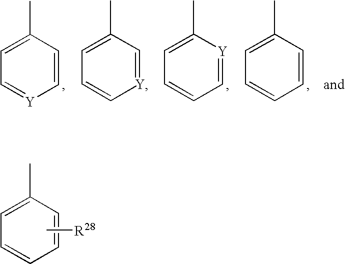
wherein Y represents N or NO, R28 is selected from the group consisting of: C1 to C4 alkyl, halo, hydroxy, NO2, amino (—NH2), —NHR30, and —N(R30)2 wherein R30 represents C1 to C6 alkyl.

In an embodiment of the invention the Pt based compound is

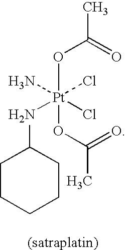

An embodiment of the invention includes a pharmaceutical composition comprising said composition and a pharmaceutically acceptable carrier. In another embodiment of the invention, satraplatin is formulated into an oral dosage form (e.g., a pill, a caplet, a tablet and a capsule).

The present invention also provides a composition or kit comprising (a) a Pt based compound represented by the structural formula: 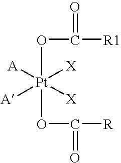
wherein A and A′ are NH₃ or a C1-C10 cyclic, straight-chain or branched-chain alkyl amine; R and R1 are selected from the group consisting of hydrogen, C1-C10 alkyl, alkenyl, aryl, aralkyl, alkylamino and alkoxy; and X is selected from the group consisting of halogen, alkyl mono-carboxylate and alkyl di-carboxylate or a Pt based compound represented by the structural formula 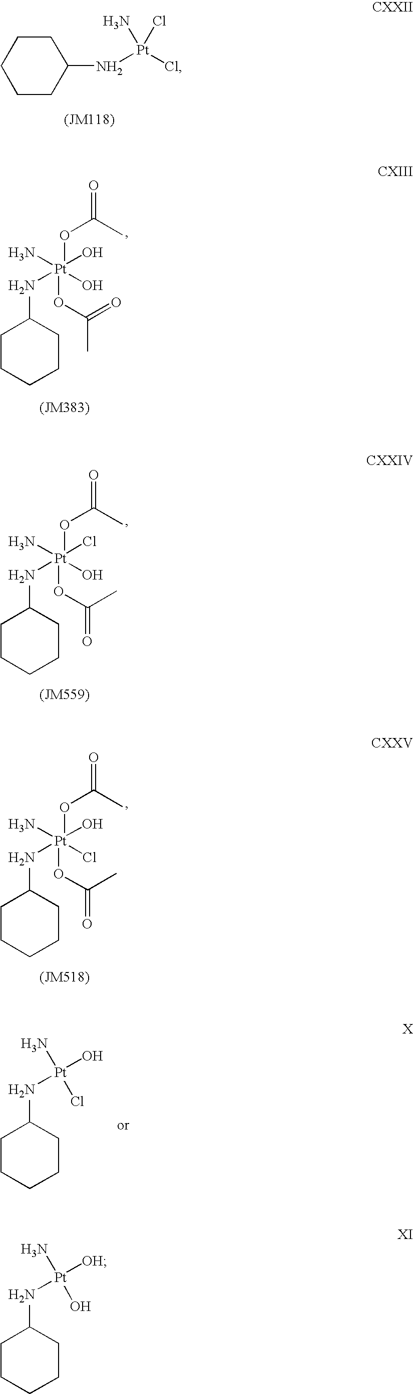
in association with

(b) another chemotherapeutic agent represented by a structural formula selected from the group consisting of: an isolated fully human monoclonal antibody that binds specifically to human IGFR1 comprising a light chain variable region comprising amino acids 20-128 of SEQ ID NO: 2 and a heavy chain variable region comprising amino acids 20-137 of SEQ ID NO:4; 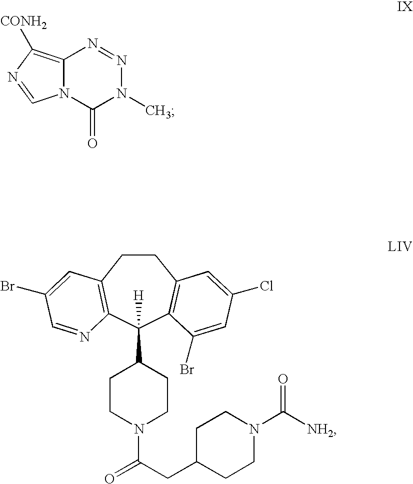
the FOLFOX regimen (i.e., a combination of (i) oxaliplatin 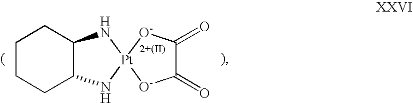
(ii) fluorouracil 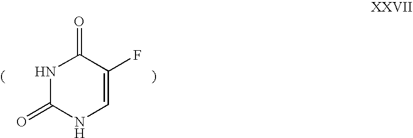
and (iii) folinic acid 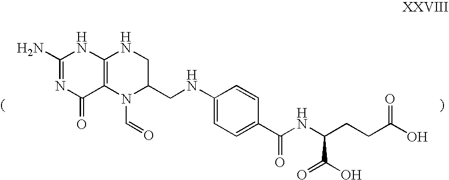
rituximab, cetuximab, HuMax-CD20, Campath-1H®, HuMax-EGFr, pertuzumab, bevacizumab, Avastin®, Ibritumomab tiuxetan, Tositumomab and Iodine I¹³¹, gemtuzumab ozogamicin and MDX-01 0. In an embodiment of the invention, the Pt based compound is 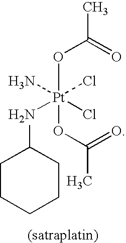
An embodiment of the invention includes a pharmaceutical composition comprising said composition and a pharmaceutically acceptable carrier. In an embodiment of the invention satraplatin is formulated into an oral dosage form (e.g., a pill, a caplet, a tablet and a capsule).

The present invention includes a method for treating or preventing cancer (e.g., prostate cancer, including hormone-refractory prostate cancer (HRPC), colon cancer, rectal cancer or colorectal cancer, non-small cell lung cancer (NSCLC), small cell lung cancer (SCLC), head & neck cancer, esophageal cancer, gastric cancer, bladder cancer, breast cancer, ovarian cancer, glioblastoma multiforme (GBM), glioma, anaplastic astrocytoma (AA) and melamona, e.g., metastatic melanoma) in a subject (e.g., a human) comprising administering, to the subject, a composition (e.g., a Pt based compound in association with another chemotherapeutic agent) according to the invention (see above). In an embodiment of the invention a composition of the invention is administered to the subject in association with an anti-cancer therapeutic procedure (e.g., surgical tumorectomy). In an embodiment of the method, satraplatin is administered to the subject orally and/or any of the other chemotherapeutic agents are administered to the subject parenterally or non-parenterally.

### DETAILED DESCRIPTION OF THE INVENTION

The present invention includes compositions comprising a platinum-based compound according the general formula I (infra) in association with one or more further cytotoxic and/or cytostatic and/or anti-hormonal and/or other cancer supportive care agents. Also within the scope of the present invention, are methods for treating any form of cancer using a composition of the invention.

The term “other chemotherapeutic agent” or “another chemotherapeutic agent”” includes cytotoxic, cytostatic, anti-hormonal or other cancer supportive care agents with the exception of satraplatin or its metabolites.

“Alkenyl” means an aliphatic hydrocarbon group containing at least one carbon-carbon double bond and which may be straight or branched and comprising about 2 to about 15 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The term “substituted alkenyl” means that the alkenyl group may be substituted by one or more substituents which may be the same or different, wherein in an embodiment, each substituent is independently selected from the group consisting of halo, alkyl. Aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkyl” means an aliphatic hydrocarbon group which may be straight or branched and comprising about 1 to about 20 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, wherein in an embodiment, each substituent is independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl, n-pentyl, heptyl, nonyl, decyl, fluoromethyl, trifluoromethyl and cyclopropylmethyl.

“Alkoxy” means an alkyl-O— group in which the alkyl group as described herein. Non-limiting examples of suitable alkoxy groups include methoxy, ethoxy, n-propoxy, isopropoxy and n-butoxy. The bond to the parent moiety is through the ether oxygen.

“Alkylthio” means an alkyl-S— group in which the alkyl group is described herein. Non-limiting examples of suitable alkylthio groups include methylthio, ethylthio, i-propylthio and heptylthio. The bond to the parent moiety is through the sulfur.

“Alkylsulfinyl” means an alkyl-S(O)— group wherein “alkyl” is as described herein. The bond to the parent moiety is through the sulfinyl.

“Alkylsulfonyl” means an alkyl-S(O₂)— group wherein “alkyl” is as described herein. The bond to the parent moiety is through the sulfonyl.

“Alkylaryl” or “aralyky” or “arylalkyl” means an alkyl-aryl- or aryl-alkyl- group in which the alkyl and aryl are as previously described. Preferred alkylaryls comprise a lower alkyl group. Non-limiting Examples of a suitable alkylaryl group is tolyl. The bond to the parent moiety is through the aryl or alkyl.

“Alkoxycarbonyl” means an alkyl-O—C(O)— group in which the alkoxy group is as described herein. Non-limiting examples of suitable alkoxycarbonyl groups include methoxycarbonyl and ethoxycarbonyl. The bond to the parent moiety is through the carbonyl.

“Alkylcarbonyl” means an alkyl-C(O)— wherein alkyl is as described herein.

“Aryl” means an aromatic monocyclic or multicyclic ring system comprising about 6 to about 14 carbon atoms. The aryl group can be optionally substituted with one or more “ring system substituents” which may be the same or different, and are as defined herein. Non-limiting examples of suitable aryl groups include phenyl and naphthyl.

“Cycloalkyl” means a non-aromatic mono- or multicyclic ring system comprising about 3 to about 10 carbon atoms. The cycloalkyl can be optionally substituted with one or more “ring system substituents” which may be the same or different. Non-limiting examples of monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like, as well as partially saturated species such as, for example, indanyl, tetrahydronaphthyl and the like.

“Cycloalkenyl” means a non-aromatic mono- or multicyclic ring system comprising about 3 to about 10 carbon atoms and one or more C to C double bonds.

“Halogen” means fluorine, chlorine, bromine, or iodine.

“Haloalkyl” means -alkyl-halogen wherein “alkyl” and “halogen” are as described herein.

“Heteroarylalkyl” means a heteroaryl substituent connected to a parent moiety via an alkyl moiety.

“Heteroalkenyl” means an alkenyl substituent connected to a heteroatom.

“Heterocyclic” means, in addition to the heteroaryl groups defined herein, saturated and unsaturated cyclic organic groups having at least one O, S and/or N atom interrupting a carbocyclic ring structure that consists of one ring or two fused rings, wherein each ring is 5-, 6-, 7- or 8-membered and may or may not have double bonds that lack delocalized pi electrons, which ring structure has from 2 to 8, preferably from 3 to 6 carbon atoms, e.g., 2- or 3-piperidinyl, 2- or 3-piperazinyl, 2- or 3-morpholinyl, or 2- or 3-thiomorpholinyl.

“Heteroaryl” means an aromatic monocyclic or multicyclic ring system comprising about 5 to about 14 ring atoms, in which one or more of the ring atoms is an element other than carbon, for example nitrogen, oxygen or sulfur, alone or in combination. The “heteroaryl” can be optionally substituted by one or more “ring system substituents” which may be the same or different. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Heteroaralkyl” means a heteroaryl-alkyl- group in which the heteroaryl and alkyl are as described herein. Preferred heteroaralkyls contain a lower alkyl group. Non-limiting examples of suitable aralkyl groups include pyridylmethyl, 2-(furan-3-yl)ethyl and quinolin-3-ylmethyl. The bond to the parent moiety is through the alkyl.

“Heteroaralkenyl” means an heteroaryl-alkenyl- group in which the heteroaryl and alkenyl are as described herein. Preferred heteroaralkenyls contain a lower alkenyl group. Non-limiting examples of suitable heteroaralkenyl groups include 2-(pyrid-3-yl)ethenyl and 2-(quinolin-3-yl)ethenyl. The bond to the parent moiety is through the alkenyl.

### Platinum Based Compounds

Any of the platinum based compounds set forth herein may be included in a composition or kit of the invention or may be used in a method of the invention (e.g., for treating or preventing cancer (infra)).

A platinum-based compound useful for the invention is any platinum-based compound set forth in U.S. Pat. No. 5,072,011: 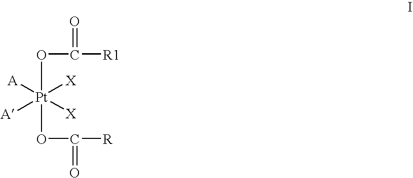
, for example, where A and A′ are NH₃ or amino, e.g., a C1-C10 alkyl amine (straight chain, branched or cyclic) such as —NH₂—C₆H₁₁; R and R1 are selected from the group consisting of hydrogen, alkyl, e.g., C1-C10 alkyl, for example —CH₃, alkenyl, aryl, aralkyl, alkylamino or alkoxy (e.g., —O—CH₃) or functionalized derivatives thereof; and X is selected from the group consisting of halogen (e.g., Cl) and alkyl mono- or di-carboxylate.

The A and A′ substituents may be the same or different. For example, in an embodiment, A and A′ are —NH₃. However, one or both of these substituents may be an —NH₃ or an amine, the nature of which can be widely varied. This includes, for example, aromatic, heteroaromatic or heterocyclic radicals, e.g., Cyclohexylamino, anilino, pyridyl, aziridine or morpholine groups, functionalized amines such as 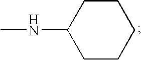
NH₂(CH₂)₃ CO₂C₂H₅; X1 CO₂-c-C₆H₁₀CH₂NH₂, X1CO₂CH₂CH₂NH₂ or the like where X1 is a lower alkyl such as methyl, ethyl, propyl, etc. A and/or A′ may also be a chelating diamine such as 1,2-diaminocyclohexane (DACH) or 2-aminomethylcyclohexylamine (AMCHA). It will be understood that when a chelating amine is used, A and A′ are joined together to complete a ring provided, however, that A and A′ do not include a cyclopentane ring when R and/or R1 are methyl.

As other specific examples of A and/or A′, there may be mentioned primary or secondary amines such as n-propylamine (n-C₃H₇NH₂), isopropylamine (i-C₃H₇ NH₂), cyclopropylamine (c-C₃H₅NH₂), cyclobutylamine (c-C₄H₇NH₂), isobutylamine (i-C₄H₉NH₂), tertiary butylamine (t-C₄H₉NH₂), neo-pentylamine, tert-amylamine, isoamylamine (i-C₅H₁₁NH₂), cyclopentylamine (c-C₅H₉NH₂), cyclohexylamine (c-C₆ H₁₁NH₂), cycloheptylamine (c-C₇H₁₃NH₂) and the corresponding secondary alkylamines, e.g., di-n-propylamine. As will be evident, the alkyl group or groups in the alkyl amine may be straight chain, branched chain or cyclic. Mixed alkyl amine groups are also encompassed.

The R and R1 substituents may also be the same or different. Representative values for the R and R1 substituents include alkyl of 1-10 carbons, straight or branched chain such as CH₃, C₂H₅, n-C₃H₇, i-C₃H₇, n-C₄H₉, t-C₄H₉, i-C₄H₉, n-C₅H₁₁, n-C₆H₁₃ or n-C₇H₁₅; cycloalkyl of 3-7 carbons such as cyclopropyl, cyclobutyl, cyclopentyl, cyclohexyl or cycloheptyl; ethenyl, propenyl or other alkenyl; phenyl, tolyl, naphthyl or similar monocyclic or dicyclic aryl; benzyl or phenethyl or like aralkyl; lower alkylamino (primary amino) of up to 6 carbons, e.g., methylamino (CH₃ NH—), ethylamino (C₂H₅NH—); and lower alkoxy of 1-6 carbons such as methoxy, ethoxy, etc. R and R1 may also be a functionalized group such as an alkoxy alkyl (e.g., methoxy-methyl), tosylamino or the like. The definition of R and R1 as given above for Formula I is intended to include such functionalized or substituted groups.

Representative of the X substituents are F, Cl, Br or other halogen, or C1-C4 alkyl mono-carboxylate such as acetate or di- carboxylate such as malonate or cyclobutane-1,1-dicarboxylate. The X substituents, like the R, R1 and A/A′ substituents, may be the same or different. They may also be combined in the form of a chelating dicarboxylate radical.

It will be appreciated that the indicated substituents for A, A′, X, R and R1 may themselves be substituted. For example, when R and/or R1 are alkyl, the alkyl may be substituted with halogen to provide a chloromethyl substituent.

In an embodiment, a subgroup of compounds within Formula (I) are those wherein A is NH3, A′ is lower alkyl (C1-C7)amino, X is chlorine and R and R1 are lower alkyl (C1-C7) or cycloalkyl of 3-7 carbons. Especially preferred are such compounds wherein A′ is cyclopentylamine (c-C₅H₉NH₂) or cyclohexylamine (c-C₆H₁₁NH₂) and R and R1 are both propyl.

Other subgroups of compounds within Formula (I) include those wherein A is NH₃, A′ is alkylamino, X is chlorine and R and R1 are lower alkylamino or lower alkoxy.

The compounds of the invention and the starting materials necessary therefor may be prepared by procedures generally known in the art. Starting compounds of the type Pt(OH)₂Cl₂ AA′ and Pt(OH)₂Cl₂ A2 are conveniently prepared by the reaction of H₂O₂ with the corresponding Pt(II) dichloro species (Brandon et al.,. J. Med. Chem. 27:861 (1984)). The synthesis of the Pt(II) dichloro species is described in the literature (see, Cleare et al., Plat. Met. Rev. 17:2 (1973) and Connors et al., Chem. Biol. Interact. 11:145 (1975)). For example, the complexes where X is halogen, may be prepared by reacting a compound Pt(OH)₂ X₂ A,A′, e.g., Pt(OH₂)Cl₂ (i-PrNH₂)₂, with the appropriate anhydride, isocyanate or pyrocarbonate. The reaction may be carried out by simply mixing the reactants together at room temperature (20°-25° C.), then chilling to crystallize out the desired product. A generally similar preparation of other Pt complexes of the formula PtCl₂ (RCO₂)₂ (i-PrNH₂)₂ where R is CF₃, CF₂ CF₃ or CF₂ CF₂ CF₃ has previously been reported but these complexes were found to be practically insoluble in water (See Cowens et al., Int. J. of Mass Spectrometry and Ion Physics, 48, 177-180 (1983)).

The complexes wherein X in Formula I is carboxylate may be prepared by acylation of the intermediate formed by hydrogen peroxide oxidation of [Pt(H₂O)₂ (R1 NH₂) (R2)2⁺] prepared in turn from Pt₂ (R1 NH₂) (R2) and silver nitrate.

In an embodiment of the invention, the compound of the general formula I is satraplatin

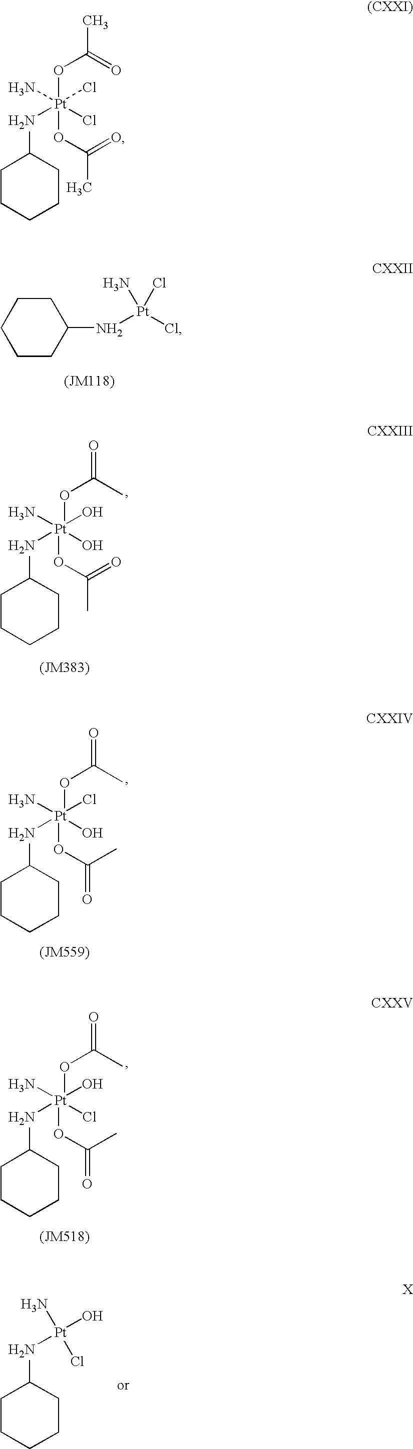

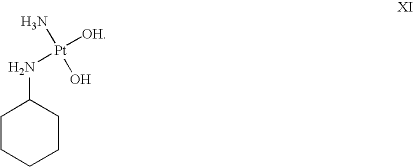

### Other Chemotherapeutic Agents

Any of the “other chemotherapeutic agents” set forth herein may be included in a composition or kit of the invention or may be used in a method of the invention (e.g., for treating or preventing cancer (infra)).

In an embodiment of the invention, a platinum-based compound represented by structural formula I (e.g., satraplatin) is provided in association with any of one or more compounds as set forth in U.S. Pat. No. 5,260,291. For example, in an embodiment of the invention, the compound is a [³H-imidazo-5,1-d]-1,2,3,5-tetrazin-4-one derivative represented by the structural formula:

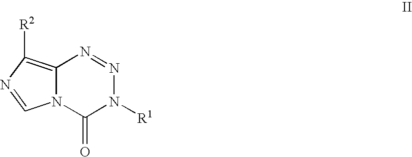

wherein R¹ represents a hydrogen atom, or a straight- or branched-chain alkyl (e.g., —CH₃), alkenyl or alkynyl group containing up to 6 carbon atoms, each such group being unsubstituted or substituted by from one to three substituents selected from halogen (i.e., bromine, iodine or, preferably, chlorine or fluorine) atoms, straight- or branched-chain alkoxy, (e.g., methoxy), alkylthio, alkylsullihinyl and alkylsulphonyl groups containing up to 4 carbon atoms, and optionally substituted phenyl groups, or R¹ represents a cycloalkyl group, and R² represents a carbamoyl group which may carry on the nitrogen atom one or two groups selected from straight- and branched-chain alkyl and alkenyl groups,each containing up to 4 carbon atoms, and cycloalkyl groups, e.g., a methylcarbamoyl or dimethylcarbamoyl group.

When the symbol R¹ represents an alkyl, alkenyl or alkynyl group substituted by two or three halogen atoms, the aforesaid halogen atoms may be the same or different. When the symbol R¹ represents an alkyl, alkenyl or alkynyl group substituted by one, two or three optionally substituted phenyl groups the optional substituents on the phenyl radical(s) may be selected from, for example, alkoxy and alkyl groups containing up to 4 carbon atoms (e.g., methoxy and/or methyl group(s)) and the nitro group; the symbol R¹ may represent, for example, a benzyl or p-methoxybenzyl group. Cycloalkyl groups within the definitions of symbols R¹ and R² contain 3 to 8, preferably 6, carbon atoms.

In an embodiment, tetrazine derivatives of general formula II are those wherein R¹ represents a straight-or branched-chain alkyl group containing from 1 to 6 carbon atoms optionally substituted by one or two halogen (preferably chlorine, fluorine or bromine) atoms or by an alkoxy group containing 1 to 4 carbon atoms (preferably methoxy) or by a phenyl group (optionally substituted by one or two alkoxy groups containing from 1 to 4 carbon atoms, preferably methoxy), or R¹ represents an alkenyl group containing 2 to 6 carbon atoms (preferably allyl) or a cyclohexyl group.

In an embodiment, tetrazine derivatives are those of general formula II wherein R¹ represents a straight- or branched-chain alkyl group containing from 1 to 6 carbon atoms, and more especially from 1 to 3 carbon atoms, unsubstituted or substituted by a halogen, preferably chlorine or fluorine, atom. In an embodiment, R¹ represents a methyl or 2-haloalkyl, e.g., 2-fluoroethyl or, preferably,2-chloroethyl, group.

In an embodiment, R² represents a carbamoyl group or a monoalkylcarbamoyl, e.g., methylcarbamoyl, or monoalkenylcarbamoyl group.

The compounds represented by structural formula II, wherein R² is as hereinbefore defined and R¹ is other than hydrogen, can be prepared by the reaction of a compound represented by structural formula: 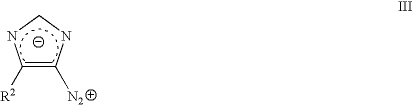
(wherein R² is as hereinbefore defined) with an isocyanate of the general formula:
R³ NCO (IV)

wherein R³ represents an alkyl, alkenyl or alkynyl group, optionally substituted by one to three substituents selected from halogen atoms, alkoxy, alkylthio, alkylsulphinyl and alkylsulphonyl groups and optionally substituted phenyl groups, or represents a cycloalkyl group, within the definition of R¹ hereinbefore recited. The reaction may be effected in the absence or presence of an anhydrous organic solvent, for example a chlorinated alkane, e.g. dichloromethane, or ethyl acetate, acetonitrile, N.-methylpyrrolid-2-one or, preferably, hexamethylphosphoramide, at a temperature between 0° C. and 70° C., e.g., at the ambient temperature. The reaction may be continued for up to 30 days. Light should be excluded from the reaction mixture.

The compounds represented by structural formula II, wherein R² is as hereinbefore defined and R¹ is other than hydrogen, can be prepared by the reaction of a compound represented by structural formula:

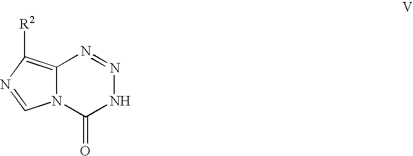

(wherein R² is as hereinbefore defined) or an alkali metal, e.g. sodium, salt thereof with a compound of the represented by structural formula:
R³ X (VI)

wherein R³ is as hereinbefore defined, and X represents the acid residue of a reactive ester, for example a halogen (e.g., chlorine) atom, or a sulphuric or sulphonic ester residue, e.g., a methoxysulphonyloxy, methanesulphonyloxy, or toluene-a-sulphonyloxy group. When R³ in a compound represented by structural formula VI represents a haloalkyl, haloalkenyl or haloalkynyl group, the acid residue of a reactive ester represented by X will be selected from those known to be not less reactive than the halogen atom substituent in R³. When X in a compound represented by structural formula VI represents a halogen atom, an alkali metal salt of the compound represented by structural formula V is preferably used and when X in a compound represented by structural formula VI represents a halogen atom and R³ is a haloalkyl, haloalkenyl or haloalkynyl group wherein the halogen atom is the same as that represented by X, an excess of the dihalo compound represented by structural formula VI is preferably used. The reaction of a compound represented by structural formula V or alkali metal salt thereof with a compound represented by structural formula VI, wherein R³ and X are as hereinbefore defined, may be carried out in a suitable anhydrous inert organic solvent, for example dichloromethane, acetonitrile or N-methylpyrrolid-2-one or mixtures thereof, at a temperature of from 0° C. to 120° C. and, when a compound represented by structural formula V is used, in the presence of an acid-binding agent, for example an alkali metal, e.g. sodium or potassium, carbonate or bicarbonate.

Compounds represented by structural formula V (i.e., compounds represented by structural formula II wherein R¹ represents a hydrogen atom and R² is as hereinbefore defined) or alkali metal salts thereof can be prepared by the reaction of a compound represented by structural formula III with a compound of the general formula:
R⁴ NCO (VII)

wherein R⁴ represents an alkali metal (e.g., sodium) atom or a protecting group such as a benzyl or p-methoxybenzyl group, followed, when R⁴ represents a protecting group, by the replacement of the protecting group by a hydrogen atom in the compound thus obtained of the general formula:

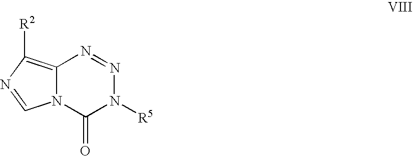

wherein R² is as hereinbefore defined, and R⁵ represents a protecting group such as a benzyl or p-methoxybenzyl group, by methods known per se. Reaction of a compound represented by structural formula III with a compound represented by structural formula VII wherein R⁴ represents a protecting group may be effected as hereinbefore described for the reaction of a compound represented by structural formula III with a compound represented by structural formula IV. Reaction of a compound represented by structural formula III with a compound represented by structural formula VII, wherein R⁴ represents an alkali metal atom, may be effected in a suitable inert organic solvent, e.g. ethanol, acetonitrile or N-methylpyrrolidone, optionally in the presence of an acid, at a temperature of from 0° C. to 120° C. The group R⁵ of compounds represented by structural formula VIII, wherein R⁵ is as hereinbefore defined, may be replaced by a hydrogen atom by methods known per se to give a compound represented by structural formula V.

In an embodiment of the invention, a platinum-based compound represented by structural formula I (e.g., satraplatin) is provided in association with temozolomide ( 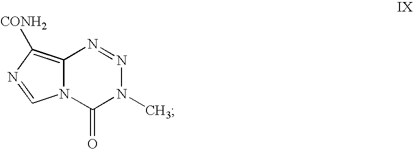
sold as Temodar® by Schering Corp.; Kenilworth, N.J.);
8-carbamoyl-3-methyl-[3H]-imidazo[5,1-d]-1,2,3,5-tetrazin-4-one;
8-carbamoyl-3-n-propyl-[3H]-imidazo[5,1-d]-1,2,3,5-tetrazin-4-one;
8-carbamoyl-3-(2-chloroethyl)-[3H]-imidazo-[5,1-d]-1,2,3,5-tetrazin-4-one;
3-(2-chloroethyl)-8-methylcarbamoyl-[3H]-imidazo[5,1-d]-1,2,3,5-tetrazin-4-one;
8-carbamoyl-3-(3-chloropropyl)-[3H]-imidazo-[5,1-d]-1,2,3,5-tetrazin-4-one;
8-carbamoyl-3-(2,3-dichloropropyl)-[3H]-imidazo[5,1-d]-1,2,3,5-tetrazin-4-one;
3-allyl-8-carbamoyl-[3H]-imidazo[5,1-d]-1,2,3,5-tetrazin-4-one;
3-(2-chloroethyl)-8-dimethylcarbamoyl-[3H]-imidazo[5,1-dl-1,2,3,5-tetrazin-4-one;
3-(2-bromoethyl)-8-carbamoyl-[3H]-imidazo-5,1-d]-1,2,3,5-tetrazin-4-one;
3-benzyl-8-carbamoyl-[3H]-imidazo[5,1-d]-1,2,3,5-tetrazin-4-one;
8-carbamoyl-3-(2-methoxyethyl)-[3H]-imidazo[5,1-d]-1,2,3,5-tetrazin-4-one;
8-carbamoyl-3-cyclohexyl-[3H]-imidazo[5,1-d]-1,2,3,5-tetrazin-4-one;
or 8-carbamoyl-3-(Wmethoxybenzyl)-[3H]imidazo[5,1-d]-1,2,3,5-tetrazin-4-one.

Other agents that, in an embodiment of the invention, are provided in association with a platinum-based compound represented by structural formula I (e.g., satraplatin), include vincristine 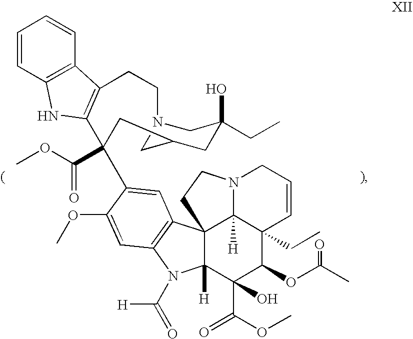
vinblastine 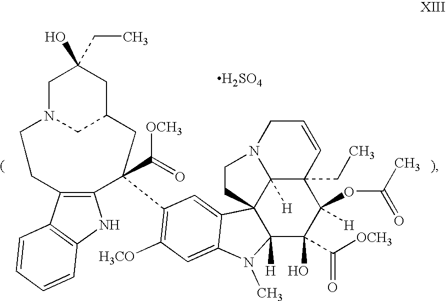
epothilone B and BMS-247550 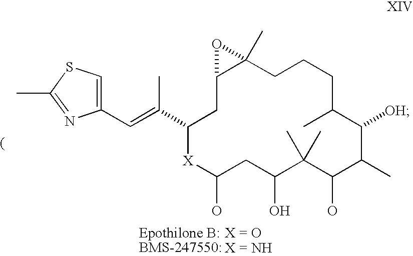
Lee et al., Clin. Cancer Res. 7(5):1429-37 (2001)), podophyllotoxins and derivatives thereof including BMS-310705 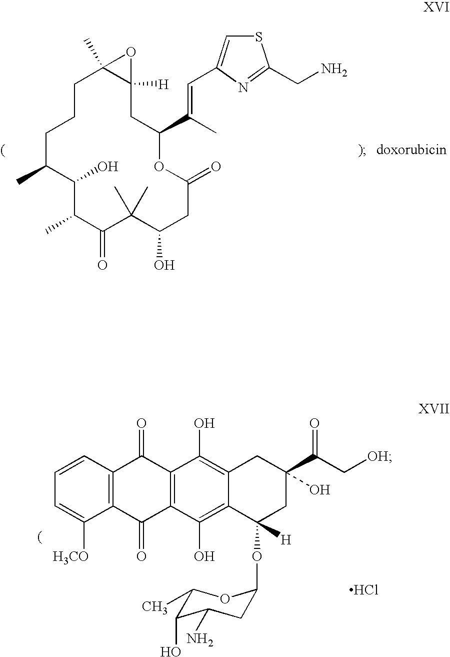
sold as Adriamycin for injection; Bedford Laboratories; Bedford, Ohio and as Doxil® (Caelyx™); Ortho Biotech Products L.P.; Raritan, N.J.).

In an embodiment of the invention, a platinum-based compound represented by structural formula I (e.g., satraplatin) is provided in association with an isolated anti-insulin-like growth factor receptor-1 (IGFR1) antibody such as any of those disclosed in any of Burtrum et. al Cancer Research 63:8912-8921(2003); in French Patent Applications FR2834990, FR2834991 and FR2834900 and in PCT Application Publication Nos. WO 03/100008; WO 03/59951; WO 04/71529; WO 03/106621; WO 04/83248; WO 04/87756 and WO 02/53596.

In a particular embodiment of the invention, a platinum-based compound represented by structural formula I (e.g., satraplatin) is provided in association with an isolated anti-insulin-like growth factor receptor-1 (IGFR1) antibody comprising a mature 19D12/15H12 Light Chain-F and a mature 19D12/15H12 heavy chain-A.

The amino acid and nucleotide sequences of the 19D12/15H12 LCF/HCA antibody chains are shown below. Dotted, underscored type indicates the signal peptide. Solid underscored type indicates the CDRs. Plain type indicates the framework regions. Mature fragments lack the signal peptide.

19D12/15H12 Light Chain-F (LCF; SEQ ID NO: 1) [symbol]
ATG TCG CCA TCA CAA CTC ATT GGG TTT CTG CTG CTC TGG GTT CCA GCC TCC [symbol]
AGG GGT GAA ATT GTG CTG ACT CAG AGC CCA GGT ACC CTG TCT GTG TCT CCA
GGC GAG AGA GCC ACC CTC TCC TGC CGG GCC AGT CAG AGC ATT GGT AGT AGC
TTA CAC TGG TAC CAG CAG AAA CCA GGT CAG GCT CCA AGG CTT CTC ATC AAG
TAT GCA TCC CAG TCC CTC TCA GGG ATC CCC GAT AGG TTC AGT GGC AGT GGA
TCT GGG ACA GAT TTC ACC CTC ACC ATC AGT AGA CTG GAG CCT GAA GAT TTC
GCA GTG TAT TAC TGT CAT CAG AGT AGT CGT TTA CCT CAC ACT TTC GGC CAA
GGG ACC AAG GTG GAG ATC AAA CGT ACA
(SEQ ID NO: 2) [symbol]
M S P S Q L I G F L L L W V P A S [symbol]
R G E I V L T Q S P G T L S V S P
G E R A T L S C R A S Q S I G S S
L H W Y Q Q K P G Q A P R L L I K
Y A S Q S L S G I P D R F S G S G
S G T D F T L T I S R L E P E D F
A V Y Y C H Q S S R L P H T F G Q
G T K V E I K R T
19D12/15H12 heavy chain-A (HCA; SEQ ID NO: 3) [symbol]
ATG GAG TTT GGG CTG AGC TGG GTT TTC CTT GTT GCT ATA TTA AAA GGT GTC [symbol]
CAG TGT GAG GTT CAG CTG GTG CAG TCT GGG GGA GGC TTG GTA AAG CCT GGG
GGG TCC CTG AGA CTC TCC TGT GCA GCC TCT GGA TTC ACC TTC AGT AGC TTT
GCT ATG CAC TGG GTT CGC CAG GCT CCA GGA AAA GGT CTG GAG TGG ATA TCA
GTT ATT GAT ACT CGT GGT GCC ACA TAC TAT GCA GAC TCC GTG AAG GGC CGA
TTC ACC ATC TCC AGA GAC AAT GCC AAG AAC TCC TTG TAT CTT CAA ATG AAC
AGC CTG AGA GCC GAG GAC ACT GCT GTG TAT TAC TGT GCA AGA CTG GGG AAC
TTC TAC TAC GGT ATG GAC GTC TGG GGC CAA GGG ACC ACG GTC ACC GTC TCC
TCA
(SEQ ID NO: 4) [symbol]
Met Glu Phe Gly Leu Ser Trp Val Phe Leu Val Ala Ile Leu Lys Gly Val [symbol]
Gln Cys Glu Val Gln Leu Val Gln Ser Gly Gly Gly Leu Val Lys Pro Gly
Gly Ser Leu Arg Leu Ser Cys Ala Ala Ser Gly Phe Thr Phe Ser Ser Phe
Ala Met His Trp Val Arg Gln Ala Pro Gly Lys Gly Leu Glu Trp Ile Ser
Val Ile Asp Thr Arg Gly Ala Thr Tyr Tyr Ala Asp Ser Val Lys Gly Arg
Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Leu Gly Asn
Phe Tyr Tyr Gly Met Asp Val Trp Gly Gln Gly Thr Thr Val Thr Val Ser
Ser

Three plasmids comprising a CMV promoter operably linked to the 15H12/19D12 LCF (κ) (variable region sequence set forth in SEQ ID NOs: 1 and 2), to the 15H12/19D12 HCA (γ4) (variable region sequence set forth in SEQ ID NOs: 3 and 4) or to the 15H12/19D12 HCA (γ1) (variable region sequence set forth in SEQ ID NOs: 3 and 4) has been deposited at the American Type Culture Collection (ATCC); 10801 University Boulevard; Manassas, Va. 20110-2209 on May 21, 2003. The deposit name and the ATCC accession numbers for the plasmids are set forth below:

CMV Promoter-15H12/19D12 HCA (γ4)—

Deposit name: “15H12/19D12 HCA (γ4)”;

ATCC accession No.: PTA-5214;
CMV Promoter-15H12/19D12 HCA (γ1)—

Deposit name: “15H12/19D12 HCA (γ4)”;

ATCC accession No.: PTA-5216;

CMV Promoter-15H12/19D12 LCF (κ)—

Deposit name: “15H12/19D12 LCF (κ)”;

ATCC accession No.: PTA-5220.

All restrictions on access to the plasmids deposited in ATCC will be removed upon grant of a patent.

The scope of the present invention includes a platinum-based compound represented by structural formula I (e.g., satraplatin) in association with an anti-insulin-like growth factor receptor-1 (IGFR1) antibody wherein the variable region of the antibody is linked to any immunoglobulin constant region. In an embodiment, the light chain variable region is linked to a κ chain constant region. In an embodiment, the heavy chain variable region is linked to a γ1, γ2, γ3 or γ4 chain constant region.

In an embodiment of the invention, a platinum-based compound represented by structural formula I (e.g., satraplatin) is provided in association with an anti-HER2 antibody; HuMax-CD20 (sold by Genmab; Copenhagen, Denmark); Campath-1H® (Riechmann et al., Nature 332:323 (1988)); HuMax-EGFr (sold by Genmab; Copenhagen, Denmark); pertuzumab (Omnitarg™, 2C4; Genentech; San Francisco, Calif.); bevacizumab (Presta et al., Cancer Res 57:4593-9 (1997); sold as Avastin® by Genentech; San Francisco, Calif.); Ibritumomab tiuxetan (sold as Zevalin® by Biogen Idec; Cambridge, Mass.); Tositumomab and Iodine I¹³¹ (sold as Bexxar® by Corixa Corp.; Seattle, Wash. and Glaxosmithkline; Philadelphia, Pa.); gemtuzumab ozogamicin (sold as Mylotarg® by Wyeth Ayerst; Madison, N.J.) or MDX-010 (Medarex; Princeton, N.J.).

In an embodiment of the invention, a platinum-based compound represented by structural formula I (e.g., satraplatin) is provided in association with 5′-deoxy-5-fluorouridine

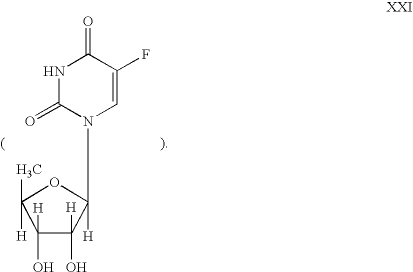

In an embodiment of the invention, a platinum-based compound represented by structural formula I (e.g., satraplatin) is provided in association with camptothecin 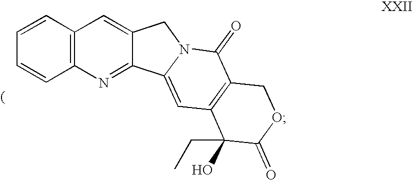
Stork et al., J. Am. Chem. Soc. 93(16): 4074-4075 (1971); Beisler et al., J. Med. Chem. 14(11): 1116-1117 (1962)) or irinotecan ( 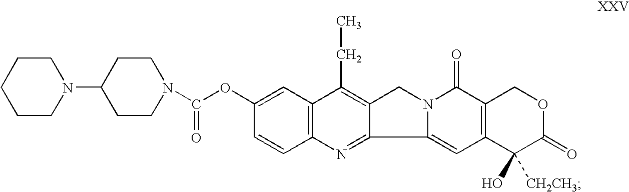
sold as Camptosar®; Pharmacia & Upjohn Co.; Kalamazoo, Mich.).

In an embodiment of the invention, a platinum-based compound represented by structural formula I (e.g., satraplatin) is provided in association with the FOLFOX regimen (oxaliplatin 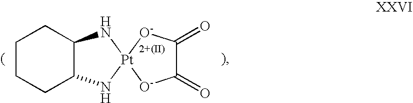
together with infusional fluorouracil 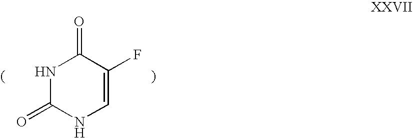
and folinic acid 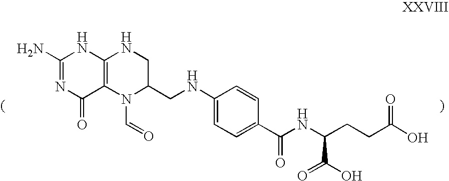
(Chaouche et al., Am. J. Clin. Oncol. 23(3):288-289 (2000);: de Gramont et al., J. Clin. Oncol. 18(16):2938-2947 (2000)). Alternatively, a platinum-based compound represented by structural formula I (e.g., satraplatin) replaces oxaliplatin in the FOLFOX regimen.

In an embodiment of the invention, a platinum-based compound represented by structural formula I (e.g., satraplatin) is provided in association with an antiestrogen such 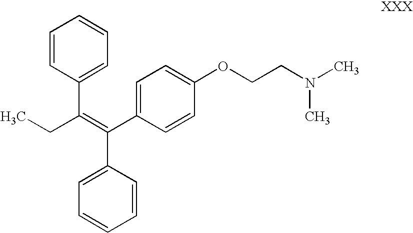
(tamoxifen; sold as Nolvadex® by AstraZeneca Pharmaceuticals LP; Wilmington, Del.) or 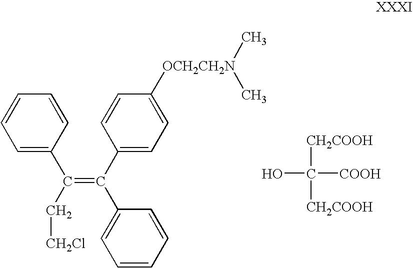
(toremifene citrate; sold as Fareston® by Shire US, Inc.; Florence, Ky.).

In an embodiment of the invention, a platinum-based compound represented by structural formula I (e.g., satraplatin) is provided in association with an aromatase inhibitor such as 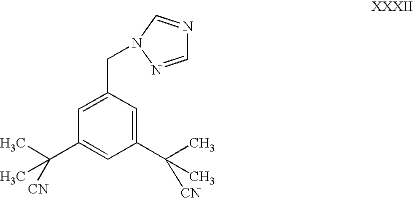
(anastrazole; sold as Arimidex® by AstraZeneca Pharmaceuticals LP; Wilmington, Del.), 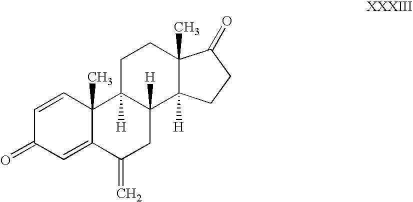
(exemestane; sold as Aromasin® by Pharmacia Corporation; Kalamazoo, Mich.) or 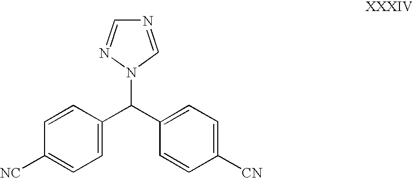
(letrozole; sold as Femara® by Novartis Pharmaceuticals Corporation; East Hanover, N.J.).

In an embodiment of the invention, a platinum-based compound represented by structural formula I (e.g., satraplatin) is provided in association with an estrogen such as DES(diethylstilbestrol), 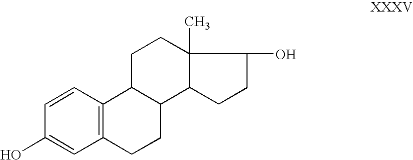
(estradiol; sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, a platinum-based compound represented by structural formula I (e.g., satraplatin) is provided in association with a LHRH (Lutenizing hormone-releasing hormone) agonist such as the acetate salt of [D-Ser(Bu t) 6,Azgly 10 ] (pyro-Glu-His-Trp-Ser-Tyr-D-Ser(Bu t)-Leu-Arg-Pro-Azgly-NH₂ acetate [C₅₉H₈₄N₁₈O₁₄·(C₂H₄O₂)ₓ where x=1 to 2.4]; 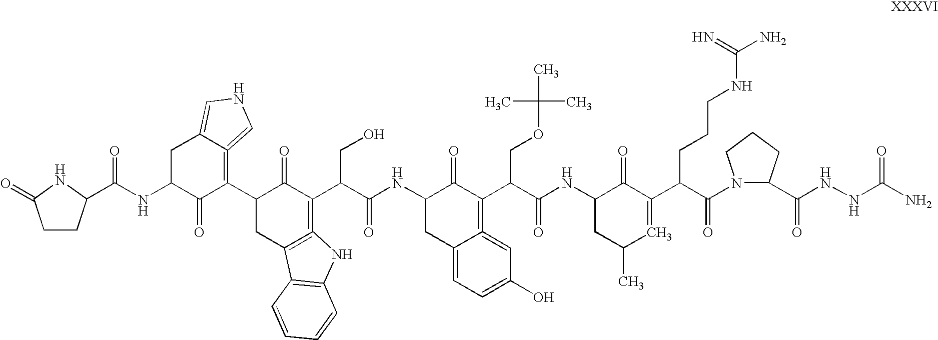
(goserelin acetate; sold as Zoladex® by AstraZeneca UK Limited; Macclesfield, England), 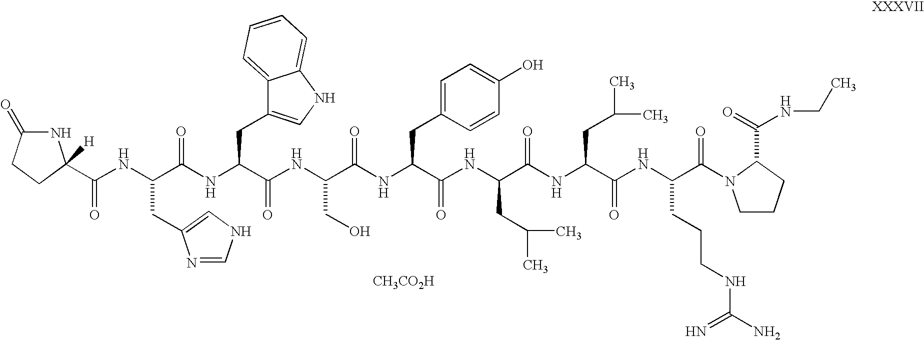
(leuprolide acetate; sold as Eligard® by Sanofi-Synthelabo Inc.; New York, N.Y.) or 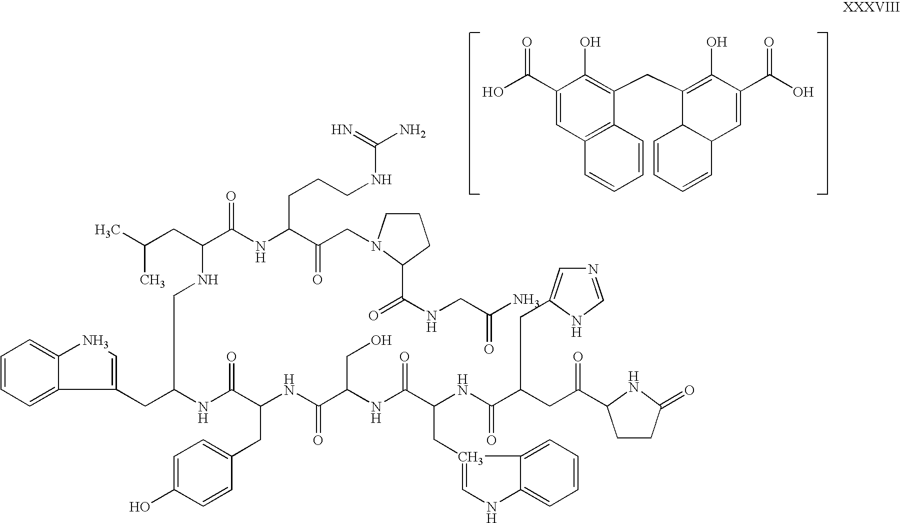
(triptorelin pamoate; sold as Trelstar® by Pharmacia Company, Kalamazoo, Mich.).

In an embodiment of the invention, a platinum-based compound represented by structural formula I (e.g., satraplatin) is provided in association with a progestational agent such as 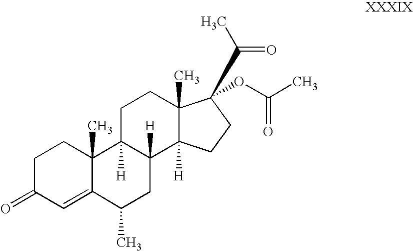
(medroxyprogesterone acetate; sold as Provera® by Pharmacia & Upjohn Co.; Kalamazoo, Mich.), 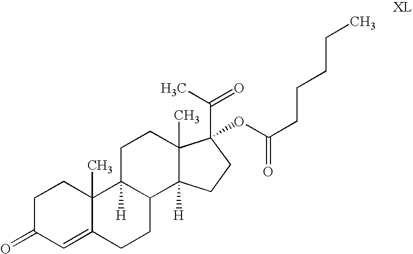
(hydroxyprogesterone caproate; 17-((1-Oxohexyl)oxy)pregn-4-ene-3,20-dione;), megestrol acetate or progestins.

In an embodiment of the invention, a platinum-based compound represented by structural formula I (e.g., satraplatin) is provided in association with selective estrogen receptor modulator (SERM) such as 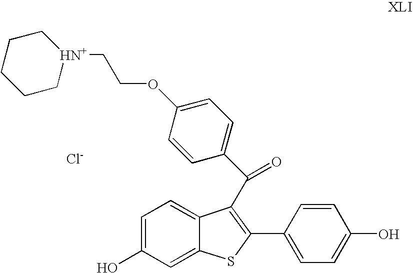
(raloxifene; sold as Evista® by Eli Lilly and Company; Indianapolis, Ind.).

In an embodiment of the invention, a platinum-based compound represented by structural formula I (e.g., satraplatin) is provided in association with an anti-androgen including, but not limited to: 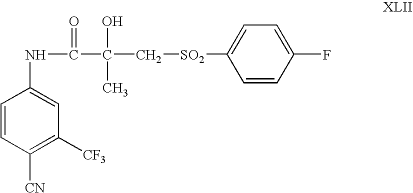
(bicalutamide; sold at CASODEX® by AstraZeneca Pharmaceuticals LP; Wilmington, Del.); 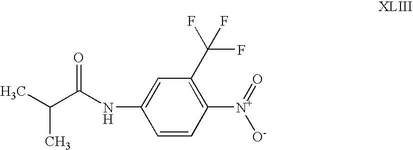
(flutamide; 2-methyl-N-[4-nitro-3 (trifluoromethyl)phenyl] propanamide; sold as Eulexin® by Schering Corporation; Kenilworth, N.J.); 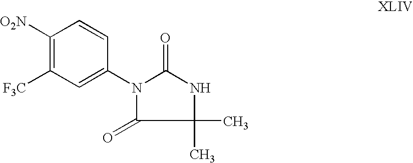
(nilutamide; sold as Nilandron® by Aventis Pharmaceuticals Inc.; Kansas City, Mo.) and 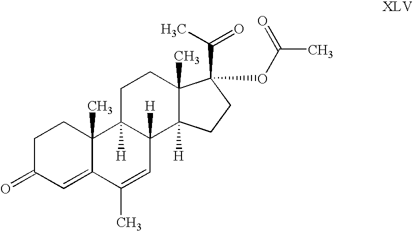
(Megestrol acetate; sold as Megace® by Bristol-Myers Squibb).

In an embodiment of the invention, a platinum-based compound represented by structural formula I (e.g., satraplatin) is provided in association with one or more inhibitors which antagonize the action of the EGF Receptor or HER2, including, but not limited to, 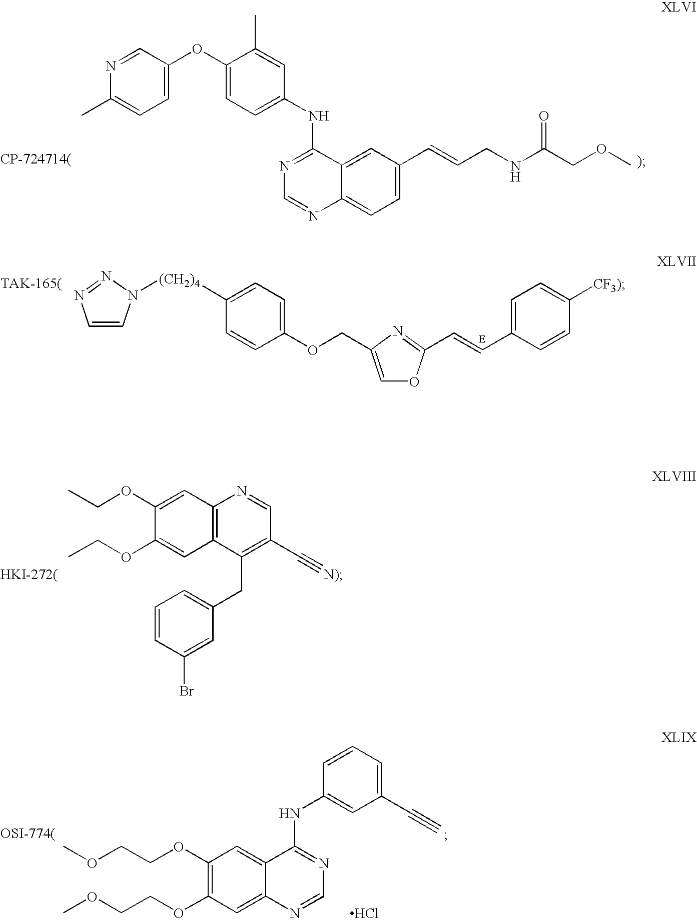
erlotinib, Hidalgo et al., J. Clin. Oncol. 19(13): 3267-3279 (2001)), Lapatanib 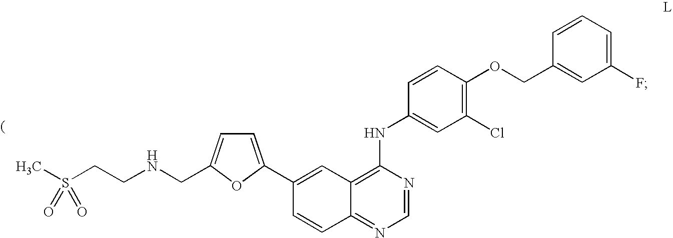
GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (Cl-1033; 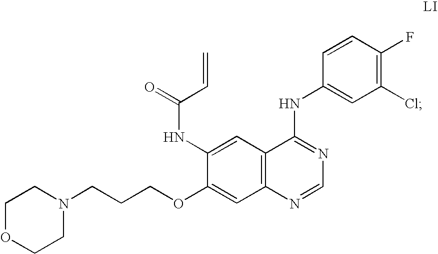
Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit Rev Oncol Hematol. 38(1):17-23 (2001)), erbitux (U.S. Pat. No. 6,217,866; IMC-C225, cetuximab; Imclone; New York, N.Y.), EKB-569 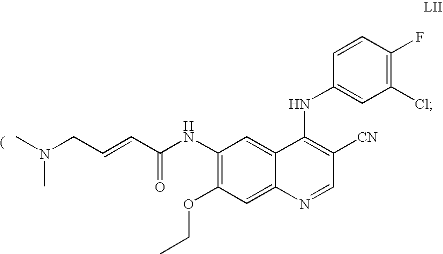
Wissner et al., J. Med. Chem. 46(1): 49-63 (2003)), PKI-166 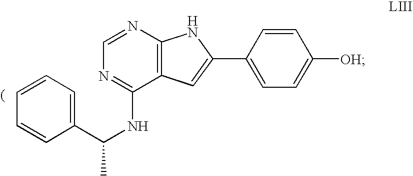
CGP-75166), GW-572016, any anti-EGFR antibody and any anti-HER2 antibody.

In an embodiment of the invention, a platinum-based compound represented by structural formula I (e.g., satraplatin) is provided in association with a farnesyl protein transferase (FPT) inhibitor including tricyclic amide compounds such as any of those disclosed in U.S. Pat. No. 5,719,148 or in U.S. Pat. No. 5,874,442. Any compound represented by formula CXIII, below, may be included in the combinations of the invention: 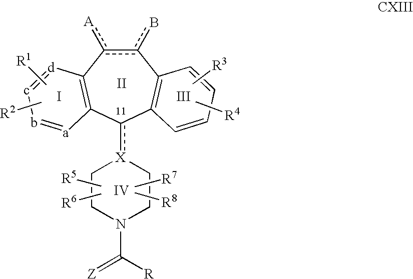
or a pharmaceutically acceptable salt or solvate thereof, wherein: one of a, b, c and d represents N or NR⁹ wherein R⁹ is O⁻, —CH₃ or —(CH₂)ₙCO₂H wherein n is 1 to 3, and the remaining a, b, c and d groups represent CR¹ or CR²; or each of a, b, c, and d are independently selected from CR¹ or CR²; each R¹ and each R² is independently selected from H, halo, —CF₃, —OR¹⁰, —COR¹⁰, —SR¹⁰, —S(O)ₜR¹¹ (wherein t is 0, 1 or 2), —SCN, —N(R¹⁰)₂, —NO₂, —OC(O)R¹⁰, —CO₂R¹⁰, —OCO₂R¹¹, —CN, —NHC(O)R¹⁰, —NHSO₂R¹⁰, —CONHR¹⁰, —CONHCH₂CH₂OH, —NR¹⁰COOR¹¹, —SR¹¹C(O)OR¹¹, 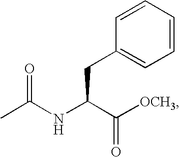
—SR¹¹N(R⁷⁵)₂ (wherein each R⁷⁵ is independently selected from H and —C(O)OR¹¹), benzotriazol-1-yloxy, tetrazol-5-ylthio, or substituted tetrazol-5-ylthio, alkynyl, alkenyl or alkyl, said alkyl or alkenyl group optionally being substituted with halo, —OR¹⁰ or —CO₂R¹⁰; R³ and R⁴ are the same or different and each independently represents H, any of the substituents of R¹ and R², or R³ and R⁴ taken together represent a saturated or unsaturated C₅-C₇ fused ring to the benzene ring; R⁵, R⁶, R⁷ and R⁸ each independently represents H, —CF₃, —COR¹⁰, alkyl or aryl, said alkyl or aryl optionally being substituted with —OR¹⁰, —SR¹⁰, —S(O)ₜR¹¹, —NR¹⁰COOR¹¹, —N(R¹⁰)₂, —NO₂, —COR¹⁰, —OCOR¹⁰, —OCO₂R¹¹, —CO₂R¹⁰, OPO₃R¹⁰ or one of R⁵, R⁶, R⁷ and R⁸ can be taken in combination with R⁴⁰ as defined below to represent —(CH₂)ᵣ— wherein r is 1 to 4 which can be substituted with lower alkyl, lower alkoxy, —CF₃ or aryl, or R⁵ is combined with R⁶ to represent ═O or ═S and/or R⁷ is combined with R⁸ to represent ═O or ═S; R¹⁰ represents H, alkyl, aryl, or aralkyl; R¹¹ represents alkyl or aryl; X represents N, CH or C, which C may contain an optional double bond, represented by the dotted line, to carbon atom 11; the dotted line between carbon atoms 5 and 6 represents an optional double bond, such that when a double bond is present, A and B independently represent —R¹⁰, halo, —OR¹¹, —OCO₂R¹¹ or —OC(O)R¹⁰, and when no double bond is present between carbon atoms 5 and 6, A and B each independently represent H₂, —(OR¹¹)₂; H and halo, dihalo, alkyl and H, (alkyl)₂, —H and —OC(O)R¹⁰, H and —OR¹⁰, ═O, aryl and H, ═NOR¹⁰ or —O—(CH₂)ₚ—O— wherein p is 2, 3 or 4; R represents R⁴⁰, R⁴², R⁴⁴, or R⁵⁴, as defined below; R⁴⁰ represents H, aryl, alkyl, cycloalkyl, alkenyl, alkynyl or -D wherein -D represents 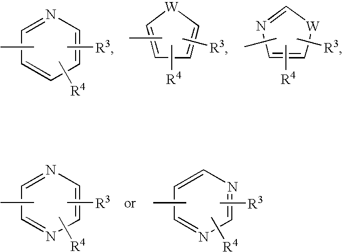
wherein R³ and R⁴ are as previously defined and W is O, S or NR¹⁰ wherein R¹⁰ is as defined above; said R⁴⁰ cycloalkyl, alkenyl and alkynyl groups being optionally substituted with from 1-3 groups selected from halo, —CON(R¹⁰)₂, aryl, —CO₂R¹⁰, —OR¹², —SR¹², —N(R¹⁰)₂, —N(R¹⁰)CO₂R¹¹, —COR¹², —NO₂ or D, wherein -D, R¹⁰ and R¹¹ are as defined above and R¹² represents R¹⁰, —(CH₂)ₘOR¹⁰ or —(CH₂)_{q}CO₂R¹⁰ wherein R¹⁰ is as previously defined, m is 1 to 4 and q is 0 to 4; said alkenyl and alkynyl R⁴⁰ groups not containing —OH, —SH or —N(R¹⁰)₂ on a carbon containing a double or triple bond respectively; or R⁴⁰ represents phenyl substituted with a group selected from —SO₂NH₂, —NHSO₂CH₃, —SO₂NHCH₃, —SO₂CH₃, —SOCH₃, —SCH₃, or —NHSO₂CF₃, preferably, said group is located in the para position of the phenyl ring; or R⁴⁰ represents a group selected from

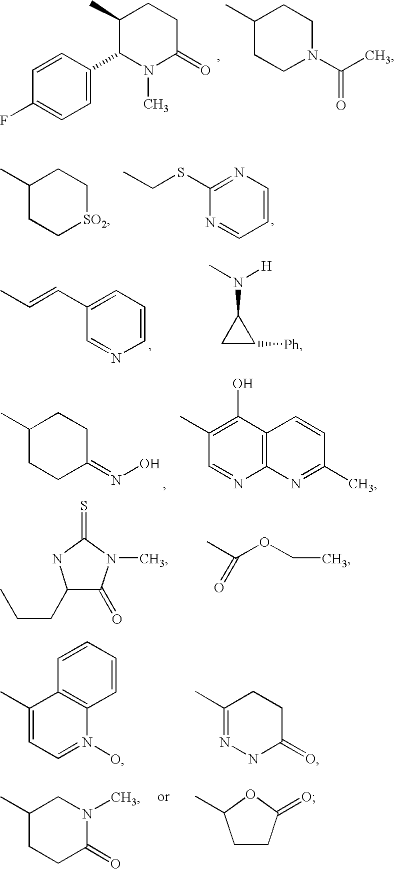

R⁴² represents 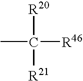
wherein R²⁰, R²¹ and R⁴⁶ are each independently selected from the group consisting of:

(1) H;

(2) —(CH₂)_{q}SC(O)CH₃ wherein q is 1 to 3;

(3) —(CH₂)_{q}OSO₂CH₃ wherein q is 1 to 3;

(4) —OH;

(5) —CS(CH₂)_{w}(substituted phenyl) wherein w is 1 to 3 and the substitutents on said substituted phenyl group are the same substitutents as described below for said substituted phenyl;

(6) —NH₂;

(7) —NHCBZ;

(8) —NHC(O)OR²² wherein R²² is an alkyl group having from 1 to 5 carbon atoms, or R²² represents phenyl substituted with 1 to 3 alkyl groups;

(9) alkyl;

(10) —(CH₂)ₖphenyl wherein k is 1 to 6;

(11) phenyl;

(12) substituted phenyl wherein the substituents are selected from the group consisting of: halo, NO₂, —OH, —OCH₃, —NH₂, —NHR²², —N(R²²)₂, alkyl, —O(CH₂)ₜphenyl (wherein t is from 1 to 3), and —O(CH₂)ₜsubstituted phenyl (wherein t is from 1 to 3);

(13) naphthyl;

(14) substituted naphthyl, wherein the substituents are as defined for substituted phenyl above;

(15) bridged polycyclic hydrocarbons having from 5 to 10 carbon atoms;

(16) cycloalkyl having from 5 to 7 carbon atoms;

(17) heteroaryl;

(18) hydroxyalkyl;

(19) substituted pyridyl or substituted pyridyl N-oxide wherein the substituents are selected from methylpyridyl, morpholinyl, imidazolyl, 1-piperidinyl, 1-(4-methylpiperazinyl), —S(O)ₜR¹¹, or any of the substituents given above for said substituted phenyl, and said substitutents are bound to a ring carbon by replacement of the hydrogen bound to said carbon;

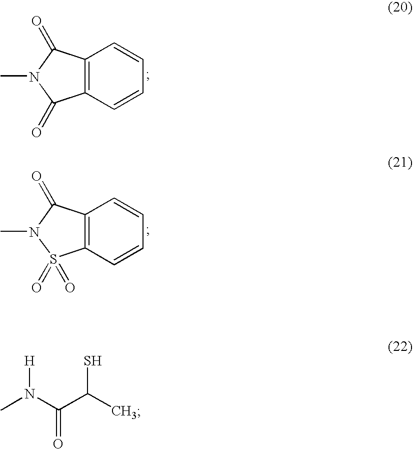

(23) —NHC(O)—(CH₂)ₖ-phenyl or —NH(O)—(CH₂)ₖ-substitued phenyl, wherein said k is as defined above;

(24) piperidine Ring V: 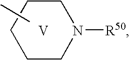
wherein R⁵⁰ represents H, alkyl, alkylcarbonyl, alkyloxycarbonyl, haloalkyl, or —C(O)NH(R¹⁰) wherein R¹⁰ is H or alkyl;; Ring V includes 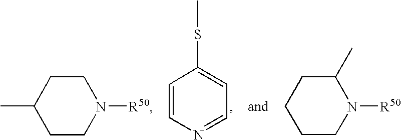
examples of Ring V include:

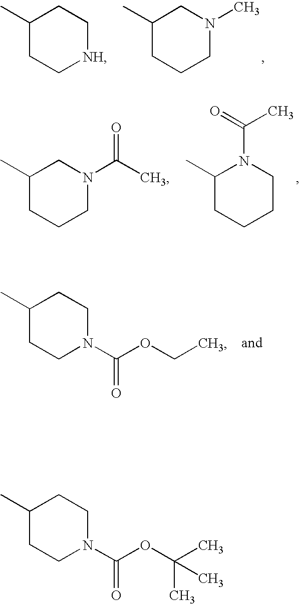

(25) —NHC(O)CH₂C₆H₅ or —NHC(O)CH₂-substituted-C₆H₅;

(26) —NHC(O)OC₆H₅;

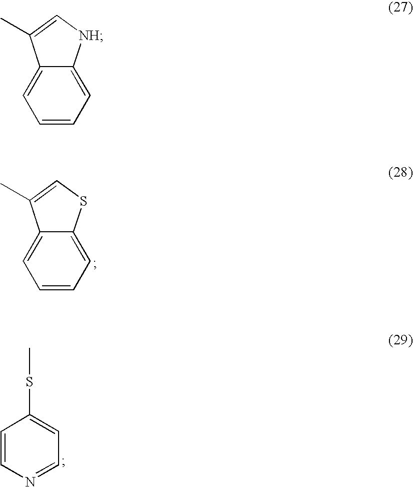

(30) —OC(O)-heteroaryl, for example

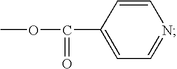

(31) —O-alkyl (e.g., —OCH₃); and

(32) —CF₃;

(33) —CN;

(34) a heterocycloalkyl group of the formula

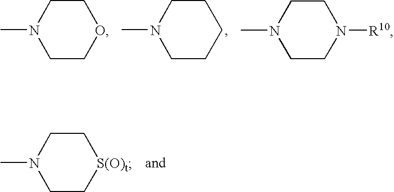

(35) a piperidinyl group of the formula 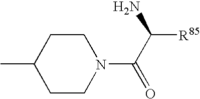
wherein R⁸⁵ is H, alkyl, or alkyl substituted by —OH or —SCH₃; or R²⁰ and R²¹ taken together form a ═O group and the remaining R⁴⁶ is as defined above; or

Two of R²⁰, R²¹ and R⁴⁶ taken together form piperidine Ring V 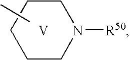
wherein R50 represents H, alkyl (e.g., methyl), alkylcarbonyl (e.g., CH3C(O)—), alkyloxycarbonyl (e.g., —C(O)O-t-C4H9, —C(O)OC2H5, and —C(O)OCH3), haloalkyl (e.g., trifluro-methyl), or —C(O)NH(R10) wherein R10 is H or alkyl; Ring V includes 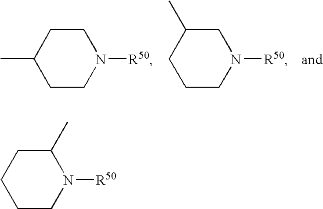
examples of Ring V include: 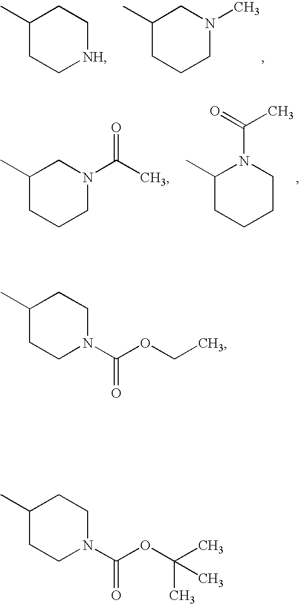
with the proviso R46, R20, and R21 are selected such that the carbon atom to which they are bound does not contain more than one heteroatom (i.e., R46, R20, and R21 are selected such that the carbon atom to which they are bound contains 0 or 1 heteroatom);R⁴⁴ represents 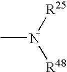
wherein R²⁵ represents heteroaryl, N-methylpiperdinyl or aryl; and R⁴⁸ represents H or alkyl; R⁵⁴ represents an N-oxide heterocyclic group of the formula (i), (ii), (iii) or (iv): 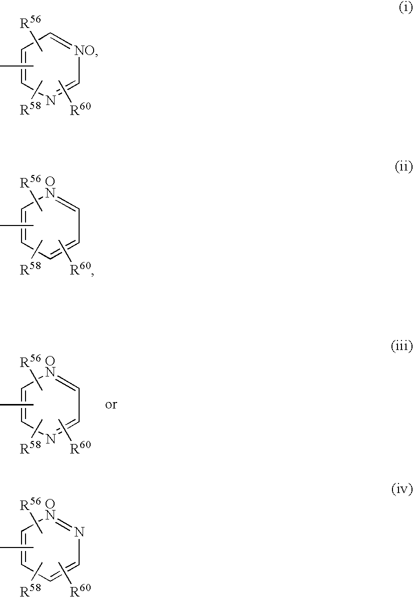
wherein R⁵⁶, R⁵⁸, and R⁶⁰ are the same or different and each is independently selected from H, halo, —CF₃, —OR¹⁰, —C(O)R¹⁰, —SR¹⁰,
—S(O)ₑR¹¹ (wherein e is 1 or 2), —N(R¹⁰)₂, —NO₂, —CO₂R¹⁰, —OCO₂R¹¹, —OCOR¹⁰, alkyl, aryl, alkenyl or alkynyl, which alkyl may be substituted with —OR¹⁰, —SR¹⁰ or —N(R¹⁰)₂ and which alkenyl may be substituted with OR¹¹ or SR¹¹; or R⁵⁴ represents an N-oxide heterocyclic group of the formula (ia), (iia), (iiia) or (iva): 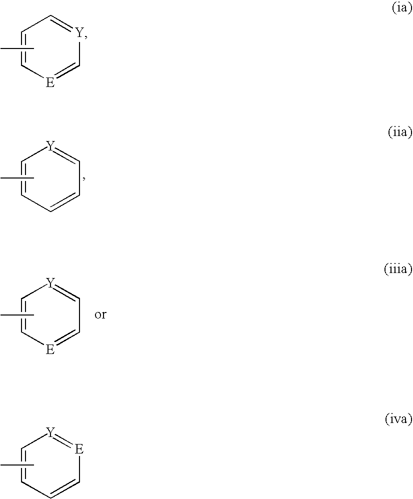
   wherein Y represents N⁺—O⁻ and E represents N; or

R⁵⁴ represents an alkyl group substituted with one of said N-oxide heterocyclic groups (i), (ii), (iii), (iv), (ia), (iia), (iiia) or (iva); Z represents O or S such that R can be taken in combination with R⁵, R⁶, R⁷ or R⁸ as defined above, or R represents R⁴⁰, R⁴², R⁴⁴ or R⁵⁴. Examples of R20, R21, and R46 for the above formulas include:

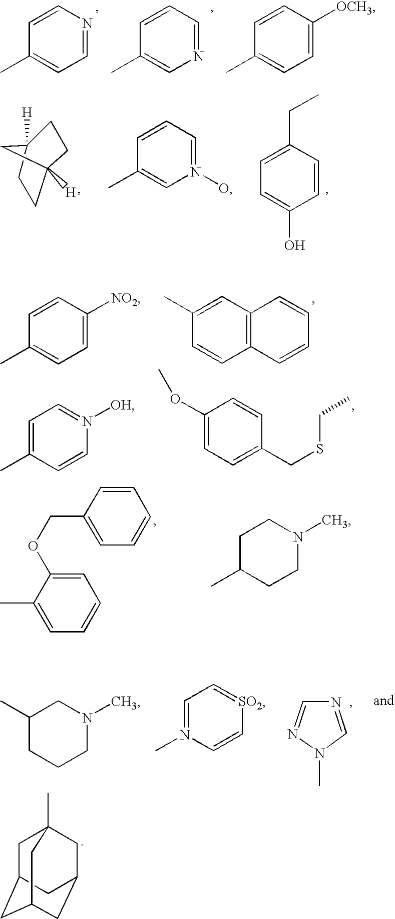

Examples of R25 groups include: 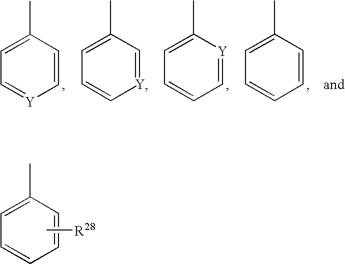
wherein Y represents N or NO, R28 is selected from the group consisting of: C1 to C4 alkyl, halo, hydroxy, NO2, amino (—NH2), —NHR30, and —N(R30)2 wherein R30 represents C1 to C6 alkyl.

In one embodiment, a platinum-based compound represented by structural formula I (e.g., satraplatin) is provided in association with: 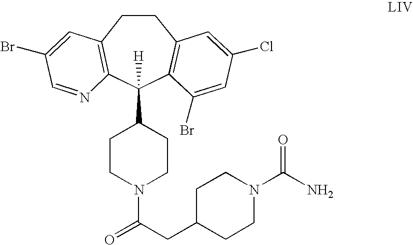
(lonafarnib; Sarasar™; Schering-Plough; Kenilworth, N.J.). In another embodiment, one of the following FPT inhibitors is provided in association with a platinum-based compound represented by structural formula I (e.g., satraplatin):

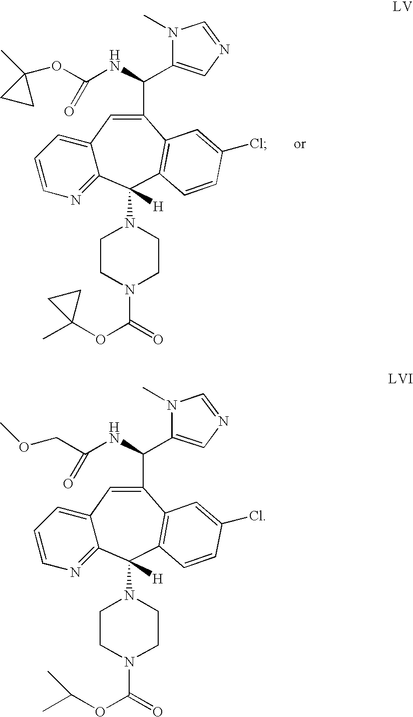

Othe FPT inhibitors, that can be provided in association with a platinum-based compound represented by structural formula I (e.g., satraplatin), include BMS-214662 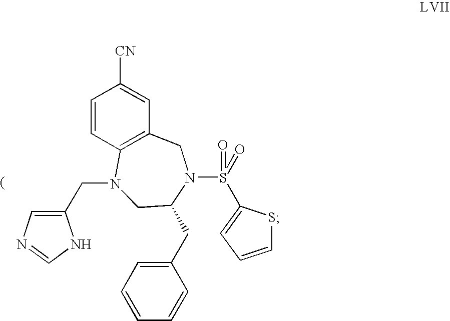
Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone];

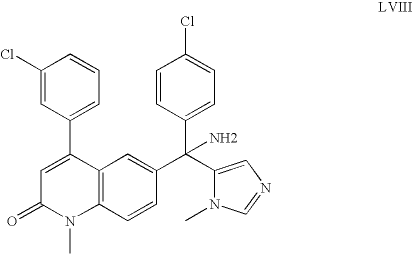

sold as Zarnestra™; Johnson & Johnson; New Brunswick, N.J.).

In an embodiment of the invention, a platinum-based compound represented by structural formula I (e.g., satraplatin) is provided in association with 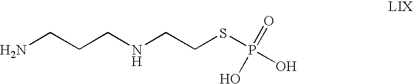
(Amifostine); 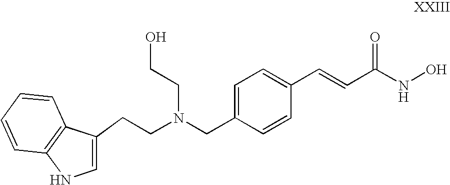
(NVP-LAQ824; Atadja et al., Cancer Research 64: 689-695 (2004)), 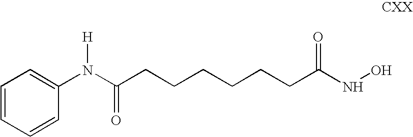
(suberoyl analide hydroxamic acid), 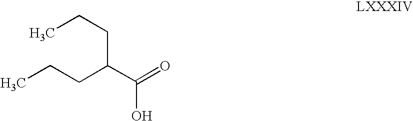
(Valproic acid; Michaelis et al., Mol. Pharmacol. 65:520-527 (2004)), 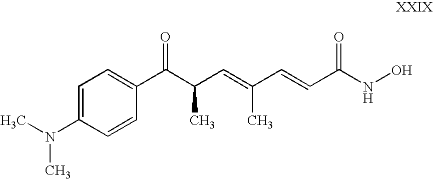
(trichostatin A), 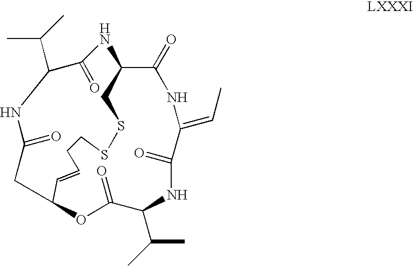
(FK-228; Furumai et al., Cancer Research 62: 4916-4921 (2002)), 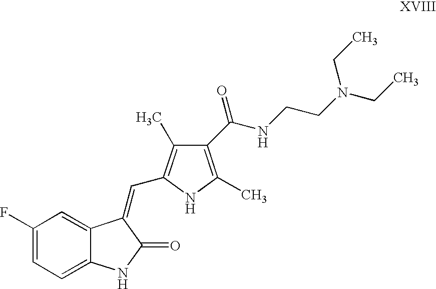
(SU11248; Mendel et al., Clin. Cancer Res. 9(1):327-37 (2003)), 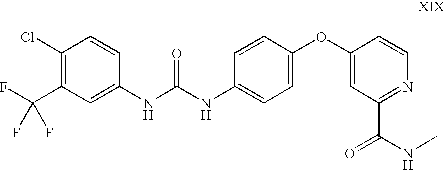
(BAY43-9006), 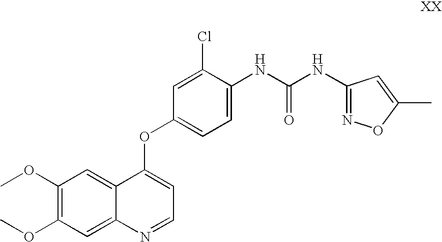
(KRN951), 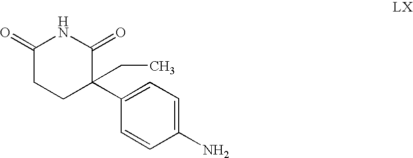
(Aminoglutethimide); 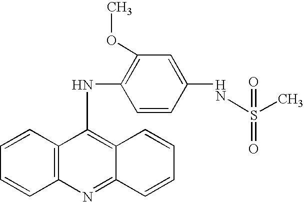
(Amsacrine); 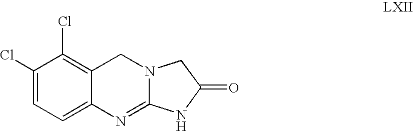
(Anagrelide); 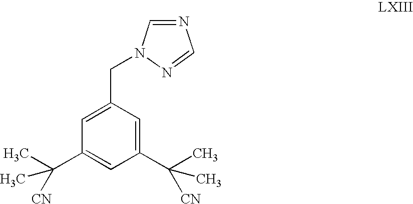
(Anastrozole; sold as Arimidex by AstraZeneca Pharmaceuticals LP; Wilmington, Del.); Asparaginase; Bacillus Calmette-Guerin (BCG) vaccine (Garrido et al., Cytobios. 90(360):47-65 (1997)); 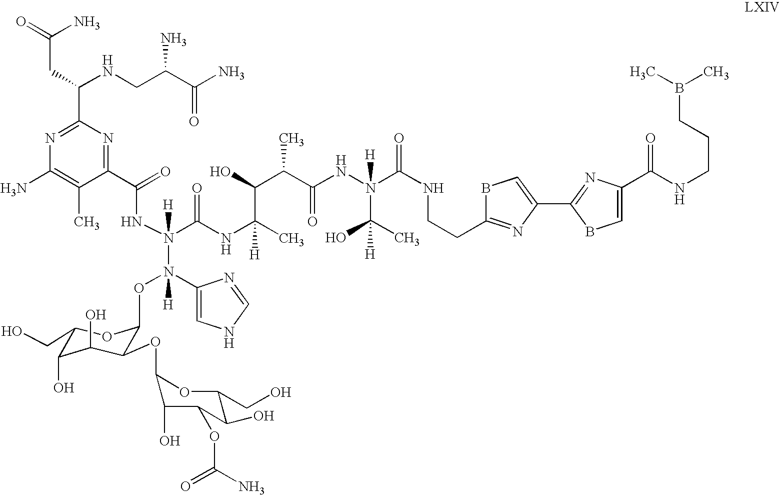
(Bleomycin); 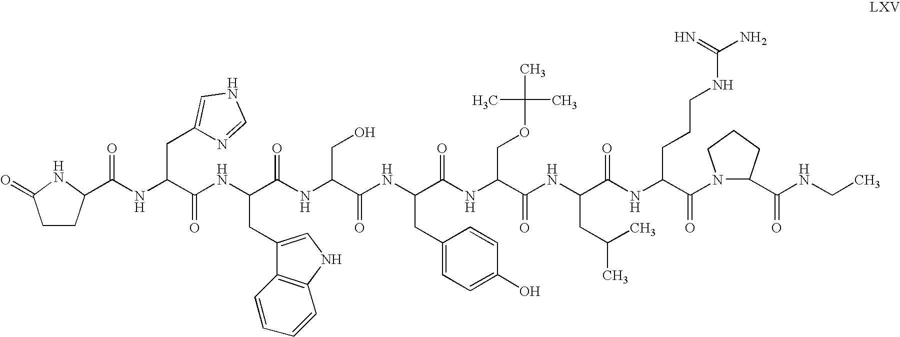
(Buserelin); 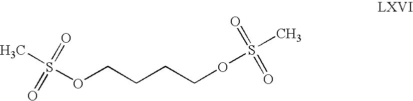
(Busulfan; 1,4-butanediol, dimethanesulfonate; sold as Busulfex® by ESP Pharma, Inc.; Edison, N.J.); 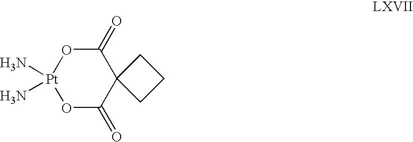
(Carboplatin; sold as Paraplatin® by Bristol-Myers Squibb; Princeton, N.J.); 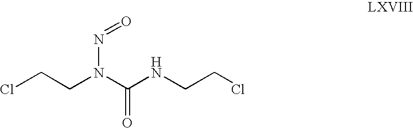
(Carmustine); 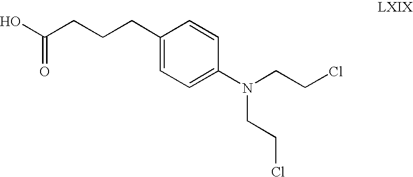
(Chlorambucil); 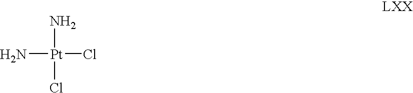
(Cisplatin); 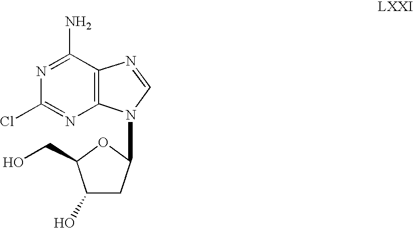
(Cladribine); 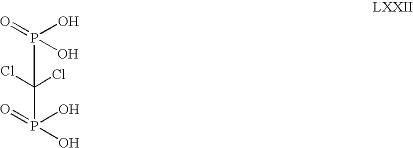
(Cyproterone); 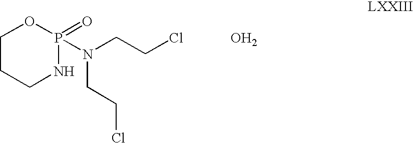
(Cyclophosphamide); 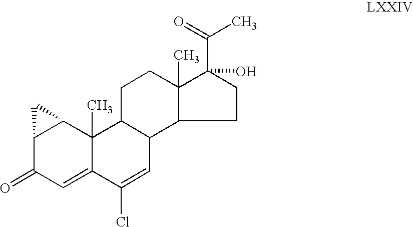
(Cyproterone); 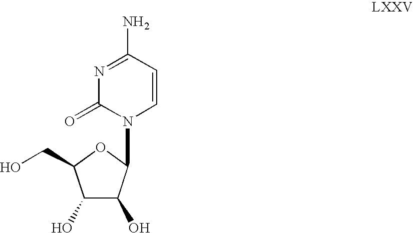
(Cytarabine); 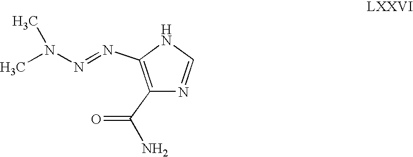
(Dacarbazine); 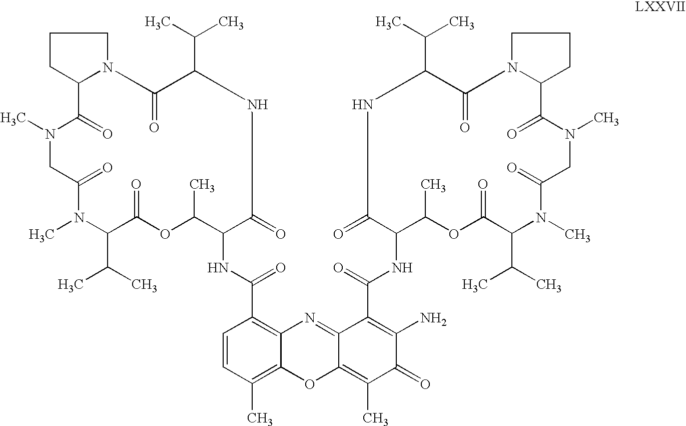
(Dactinomycin); 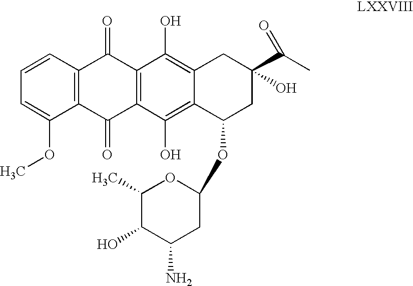
(Daunorubicin); 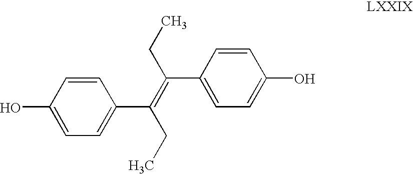
(Diethylstilbestrol); 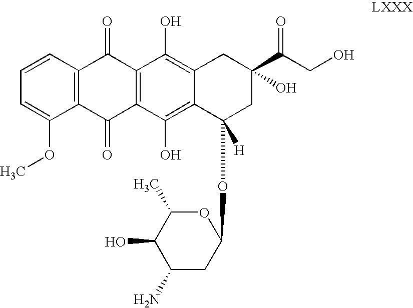
(Epirubicin);; 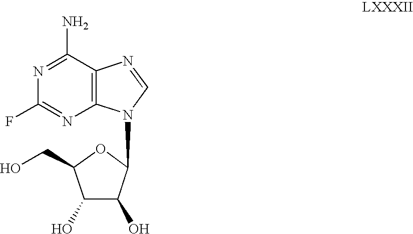
(Fludarabine); 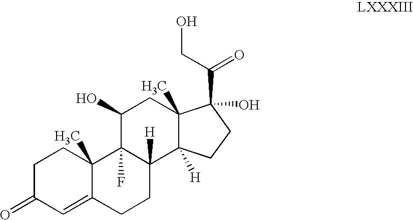
(Fludrocortisone); 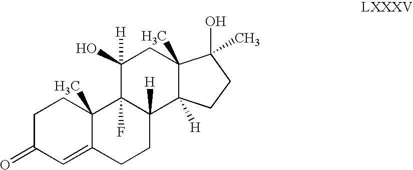
(Fluoxymesterone); 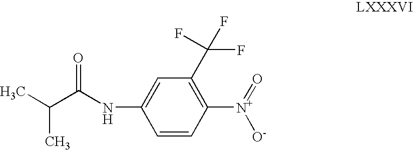
(Flutamide); 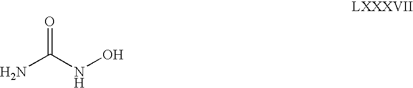
(Hydroxyurea); 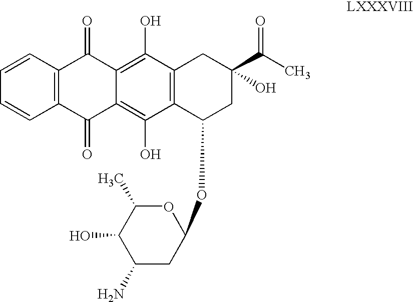
(Idarubicin); 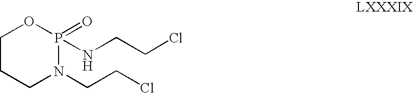
(Ifosfamide): 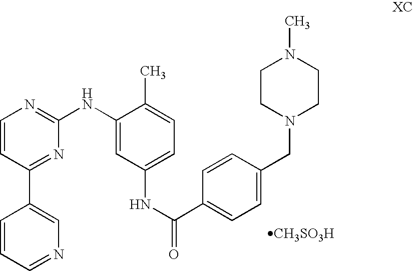
(Imatinib; sold as Gleevec® by Novartis Pharmaceuticals Corporation; East Hanover, N.J.); 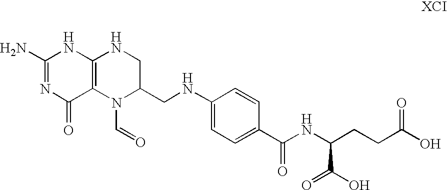
(Leucovorin); 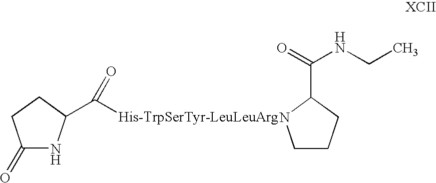
(Leuprolide); 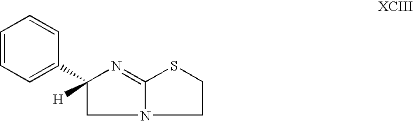
(Levamisole), 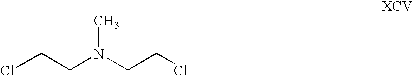
(Lomustine); 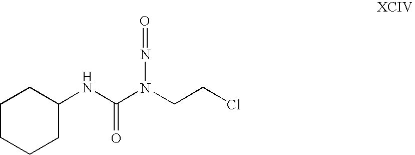
(Mechlorethamine); 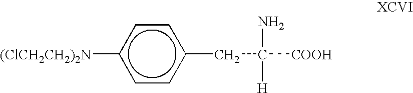
(Melphalan; sold as Aleran® by Celgene Corporation; Warren, N.J.); 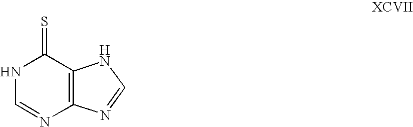
(Mercaptopurine); 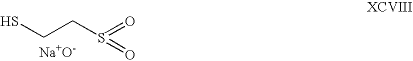
(Mesna); 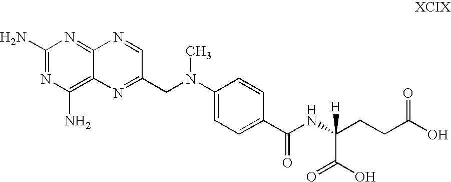
(Methotrexate); 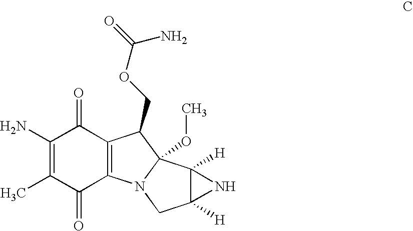
(Mitomycin); 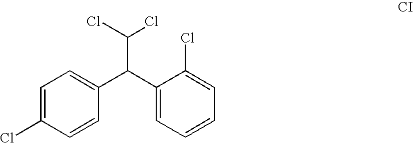
(Mitotane); 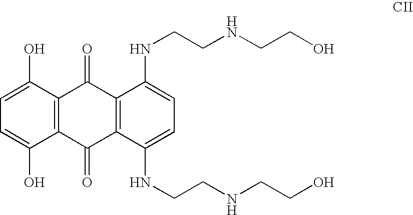
(Mitoxantrone); 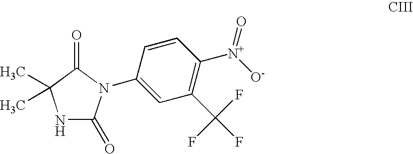
(Nilutamide); octreotide (L-Cysteinamide, D-phenylalanyl-L-cysteinyl-L-phenylalanyl-D-tryptophyl-L-lysyl-L-threonyl-N-[2-hydroxy-1-(hydroxymethyl) propyl]-, cyclic (2_{—}7)-disulfide; [R R*, R*)];

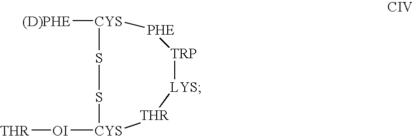

Katz et al., Clin Pharm. 8(4):255-73 (1989); sold as Sandostatin LAR® Depot; Novartis Pharm. Corp; E. Hanover, N.J.); oxaliplatin ( 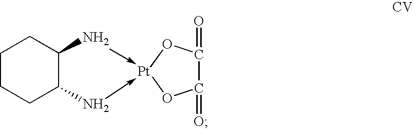
sold as Eloxatin™ by Sanofi-Synthelabo Inc.; New York, N.Y.); 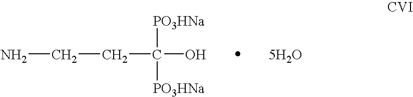
(Pamidronate; sold as Aredia® by Novartis Pharmaceuticals Corporation; East Hanover, N.J.); 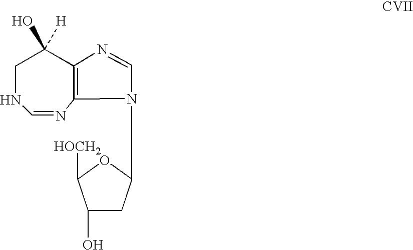
(Pentostatin; sold as Nipent® by Supergen; Dublin, Calif.); 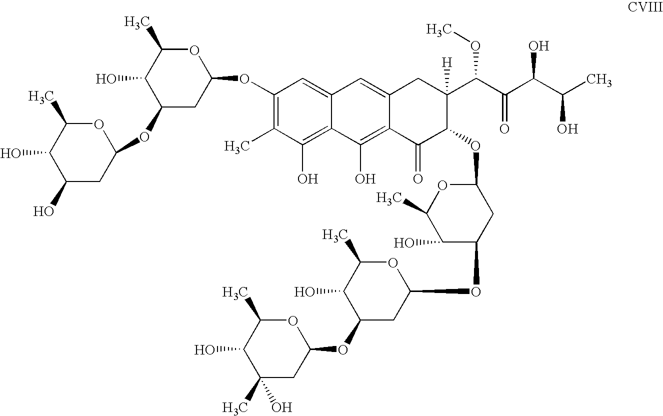
(Plicamycin); 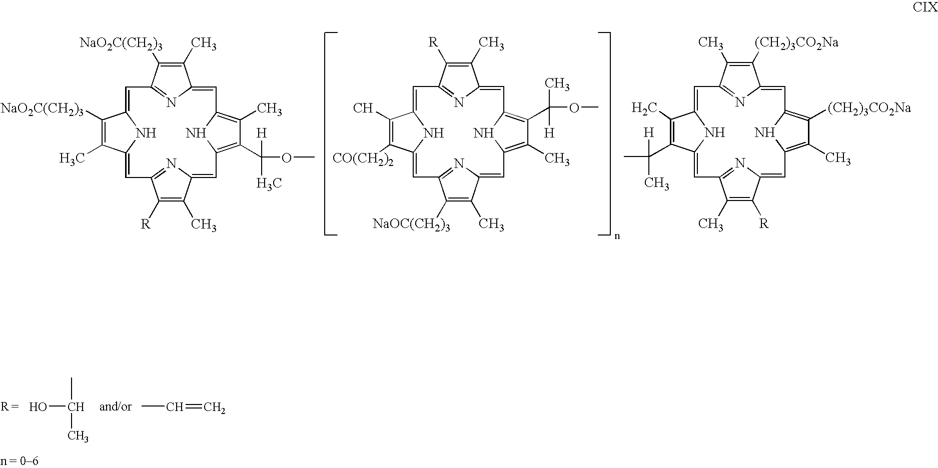
(Porfimer; sold as Photofrin® by Axcan Scandipharm Inc.; Birmingham, Ala.); 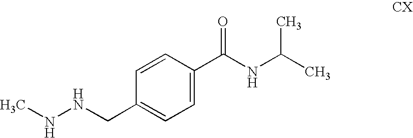
(Procarbazine); 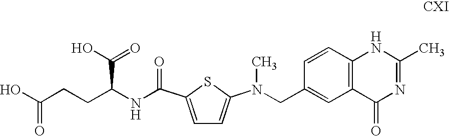
(Raltitrexed); Rituximab (sold as Rituxan® by Genentech, Inc.; South San Francisco, Calif.); 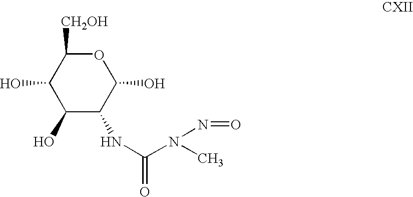
(Streptozocin); 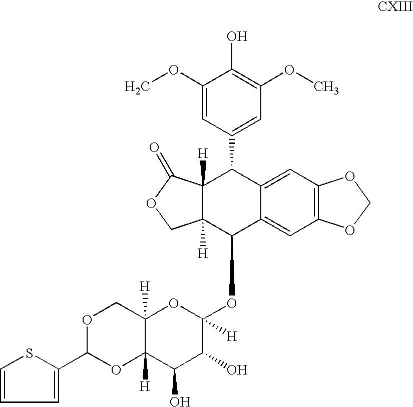
(Teniposide); 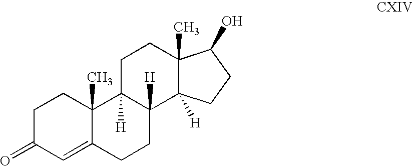
(Testosterone); 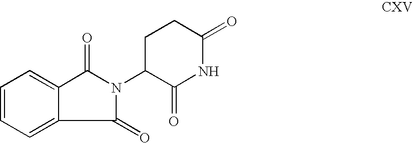
(Thalidomide); 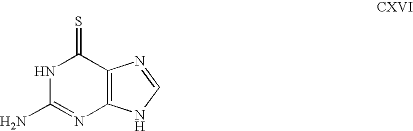
(Thioguanine); 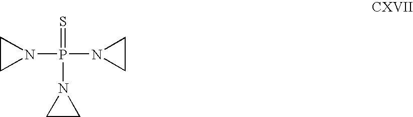
(Thiotepa); 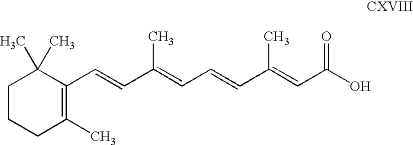
(Tretinoin); or 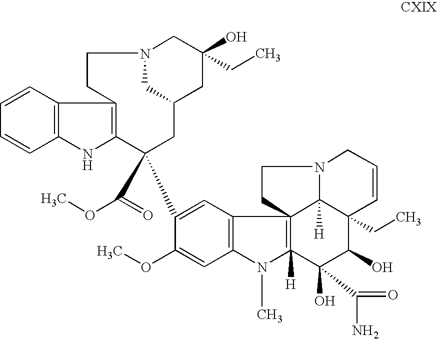
(Vindesine).

In an embodiment of the invention, a platinum-based compound represented by structural formula I (e.g., satraplatin) is provided in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha.

The term “interferon alpha” as used herein means the family of highly homologous species-specific proteins that inhibit cellular proliferation and modulate immune response. Typical suitable interferon-alphas include, but are not limited to, recombinant interferon alpha-2b, recombinant interferon alpha-2a, recombinant interferon alpha-2c, alpha 2 interferon, interferon alpha-n1 (INS), a purified blend of natural alpha interferons, a consensus alpha interferon such as those described in U.S. Pat. Nos. 4, 897,471 and 4,695,623 (especially Examples 7, 8 or 9 thereof), or interferon alpha-n3, a mixture of natural alpha interferons.

Interferon alfa-2a is sold as ROFERON-A® by Hoffmann-La Roche (Nutley, N.J).

Interferon alfa-2b is sold as INTRON-A® by Schering Corporation (Kenilworth, N.J.). The manufacture of interferon alpha 2b is described, for example, in U.S. Pat. No. 4,530,901.

Interferon alfa-n3 is a mixture of natural interferons sold as ALFERON N INJECTION® by Hemispherx Biopharma, Inc. (Philadelphia, Pa.).

Interferon alfa-n1 (INS) is a mixture of natural interferons sold as WELLFERON® by Glaxo-Smith-Kline (Research Triangle Park, N.C.).

Consensus interferon is sold as INFERGEN® by Intermune, Inc. (Brisbane, Calif.).

Interferon alfa-2c is sold as BEROFOR® by Boehringer Ingelheim Pharmaceutical, Inc. (Ridgefield, Conn.).

A purified blend of natural interferons is sold as SUMIFERON® by Sumitomo; Tokyo, Japan.

The term “pegylated interferon alpha” as used herein means polyethylene glycol modified conjugates of interferon alpha, preferably interferon alpha-2a and alpha-2b. The preferred polyethylene-glycol-interferon alpha-2b conjugate is PEG 12000-interferon alpha-2b. The phrases “12,000 molecular weight polyethylene glycol conjugated interferon alpha” and “PEG 12000-IFN alpha” as used herein include conjugates such as are prepared according to the methods of International Application No. WO 95/13090 and containing urethane linkages between the interferon alpha-2a or -2b amino groups and polyethylene glycol having an average molecular weight of 12000. The pegylated inteferon alpha, PEG 12000-IFN-alpha-2b is available from Schering-Plough Research Institute, Kenilworth, N.J.

The preferred PEG 12000-interferon alpha-2b can be prepared by attaching a PEG polymer to the epsilon amino group of a lysine residue in the interferon alpha-2b molecule. A single PEG 12000 molecule can be conjugated to free amino groups on an IFN alpha-2b molecule via a urethane linkage. This conjugate is characterized by the molecular weight of PEG 12000 attached. The PEG 12000-IFN alpha-2b conjugate can be formulated as a lyophilized powder for injection.

Pegylated interferon alfa-2b is sold as PEG-INTRON® by Schering Corporation (Kenilworth, N.J.).

Pegylated interferon-alfa-2a is sold as PEGASYS® by Hoffmann-La Roche (Nutley, N.J).

Other interferon alpha conjugates can be prepared by coupling an interferon alpha to a water-soluble polymer. A non-limiting list of such polymers includes other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described, for example, in U.S. Pat. Nos. 4,766,106, 4,917, 888, European Patent Application No. 0 236 987 or 0 593 868 or International Publication No. WO 95/13090.

Pharmaceutical compositions of pegylated interferon alpha suitable for parenteral administration can be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g., NaCl), preservatives (e.g., thimerosol, cresol or benzyl alcohol), and surfactants (e.g., tween or polysorbates) in sterile water for injection. The pegylated interferon alpha can be stored as lyophilized powder under refrigeration at 2°-8° C. The reconstituted aqueous solutions are stable when stored between 2° and 8° C. and used within 24 hours of reconstitution. See for example U.S. Pat. Nos., 4,492,537; 5,762,923 and 5, 766,582. The reconstituted aqueous solutions may also be stored in prefilled, multi-dose syringes such as those useful for delivery of drugs such as insulin. Typical, suitable syringes include systems comprising a prefilled vial attached to a pen-type syringe such as the NOVOLET® Novo Pen available from Novo Nordisk or the REDIPEN®, available from Schering Corporation, Kenilworth, N.J. Other syringe systems include a pen-type syringe comprising a glass cartridge containing a diluent and lyophilized pegylated interferon alpha powder in a separate compartment.

The scope of the present invention also includes compositions comprising a platinum-based compound represented by structural formula I (e.g., satraplatin) in association with one or more other chemotherapeutic agents (e.g., as described herein) and in association with one or more antiemetics including, but not limited to, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron ( sold as Zofran® by by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention also includes methods for treating or preventing cancer in a subject by administering a platinum-based compound represented by structural formula I (e.g., satraplatin) in association with one or more other chemotherapeutic agents (e.g., as described herein) and in association with one or more antiemetics.

### Pharmaceutical Compositions

A combination, or any component thereof, of the invention can be incorporated into a pharmaceutical composition, along with a pharmaceutically acceptable carrier, suitable for administration to a subject in vivo. The scope of the present invention includes pharmaceutical compositions which may be administered to a subject by any route, such as a non-parenteral (e.g., oral, ocular, topical or pulmonary (inhalation)) or a parenteral route (e.g., intratumoral injection, intravenous injection, intraarterial injection, subcutaneous injection or intramuscular injection). In one embodiment, a pharmaceutical composition of the invention includes a platinum-based compound represented by structural formula I (e.g., satraplatin) in association with one or more other chemotherapeutic agents (e.g., as described herein) and a pharmaceutically acceptable carrier.

As stated above, the combinations of the invention include a platinum-based compound represented by structural formula I (e.g., satraplatin) component and another chemotherapeutic agent component “in association” with one another. The term “in association” indicates that the components of the combinations of the invention can be formulated into a single composition (e.g., for simultaneous delivery) or formulated separately into two or more compositions (e.g., a kit). For example, the scope of the present invention includes combinations comprising a platinum-based compound represented by structural formula I (e.g., satraplatin) formulated for parenteral administration (e.g., intravenous) to a subject and another chemotherapeutic agent formulated for oral delivery (e.g., pill, tablet, capsule). Alternatively, both components of the combination can be formulated, separately or together, for parenteral delivery or non-parenteral delivery (e.g., oral).

For general information concerning formulations, see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker.

Pharmaceutically acceptable carriers are conventional and very well known in the art. Examples include aqueous and nonaqueous carriers, stabilizers, antioxidants, solvents, dispersion media, coatings, antimicrobial agents, buffers, serum proteins, isotonic and absorption delaying agents, and the like that are physiologically compatible. In an embodiment, the carrier is suitable for injection into a subject's body.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Stabilizers, such as α, α-trehalose dihydrate may be included for stabilizing an anti-cancer antibody from degrading effects of dessication or freeze-drying.

Examples of pharmaceutically-acceptable antioxidants include: water soluble antioxidants such as ascorbic acid, cysteine hydrochloride, sodium bisulfate, sodium metabisulfite, sodium sulfite and the like; and oil-soluble antioxidants such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Prevention of the contamination of a composition of the invention with microorganisms may be ensured both by sterilization procedures, and by the inclusion of various antimicrobial agents such as EDTA, EGTA, paraben, chlorobutanol, phenol sorbic acid, and the like.

Buffers that may be included in the pharmaceutical compositions of the invention include L-histidine based buffers, phosphate based buffers (e.g., phosphate buffered saline, pH˜7), sorbate based buffers or glycine-based buffers.

Serum proteins which may be included in the pharmaceutical compositions of the invention may include human serum albumin.

Isotonic agents, such as sugars, ethanol, polyalcohols (e.g., glycerol, propylene glycol, liquid polyethylene glycol, mannitol or sorbitol), sodium citrate or sodium chloride (e.g., buffered saline) may also be included in the pharmaceutical compositions of the invention.

Prolonged absorption of an injectable pharmaceutical form may be brought about by the inclusion of agents which delay absorption such as aluminum monostearate and/or gelatin.

Dispersions can also be prepared in glycerol, liquid polyethylene glycols, and mixtures thereof and in oils.

Pharmaceutically acceptable carriers include sterile aqueous solutions or dispersions and sterile powders for the extemporaneous preparation of sterile injectable solutions or dispersions. The use of such media and agents for pharmaceutically active substances is well known in the art.

Sterile injectable solutions can be prepared by incorporating a combination of the invention or any component thereof (e.g., a platinum-based compound represented by structural formula I (e.g., satraplatin) and/or another chemotherapeutic agent), in the required amount, in an appropriate solvent, optionally with one or a combination of ingredients enumerated above, as required, followed by sterilization microfiltration. Generally, dispersions are prepared by incorporating the active ingredient (e.g., a platinum-based compound represented by structural formula I (e.g., satraplatin) and/or another chemotherapeutic agent) into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional, desired ingredient from a previously sterile-filtered solution thereof.

A combination or the invention or any component thereof (e.g., a platinum-based compound represented by structural formula I (e.g., satraplatin) and/or another chemotherapeutic agent) may also be orally administered. Pharmaceutical compositions for oral administration may include additives and carriers such as starch (e.g., lactose, starch, sucrose, cellulose, magnesium stearate, dicalcium phosphate, calcium sulfate, talc, mannitol, ethyl alcohol (liquid forms) and the like), sugars (e.g., lactose), talc, lactose, stearate, magnesium carbonate or calcium phosphate. Moreover, when desired or needed, suitable binders (e.g., starch, gelatin, natural sugars, corn sweeteners, natural and synthetic gums such as acacia, sodium alginate, carboxymethyl-cellulose, polyethylene glycol or waxes), lubricants (e.g., stearic acid, boric acid, sodium benzoate, sodium acetate, sodium chloride, and the like), disintegrating agents (e.g., starch, methylcellulose, guar gum and the like) and coloring agents (e.g., food grade dyes) may also be incorporated in the mixture. Sweetening and flavoring agents and preservatives may also be included where appropriate. In order to ensure that oral compositions are well tolerated by the patient's digestive system, mucus formers or resins may be included. It may also be desirable to improve tolerance by formulating in a capsule which is insoluble in the gastric juices. An exemplary pharmaceutical composition of this invention in the form of a capsule is prepared by filling a standard two-piece hard gelatin capsule with the combination of the invention or any component thereof in powdered form, lactose, talc and magnesium stearate. Powers and tablets may comprise, for example, from about 5 to about 95 percent active ingredient.

A combination of the invention or any component thereof may also be included in a pharmaceutical composition for topical administration. Formulations suitable for topical administration include liquid or semi-liquid preparations suitable for penetration through the skin to the site where treatment is required, such as liniments, lotions, creams, ointments or pastes, and drops suitable for administration to the eye, ear or nose.

Drops according to the present invention may comprise sterile aqueous or oily solutions or suspensions and may be prepared by dissolving the combination of the invention or any component thereof in a suitable aqueous solution of a bactericidal and/or fungicidal agent and/or any other suitable preservative, and preferably including a surface active agent. The resulting solution may then be clarified by filtration.

Lotions according to the present invention include those suitable for application to the skin or eye. An eye lotion may comprise a sterile, aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the combination of the invention or any component thereof in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

A combination of the invention or any component thereof may also be administered by inhalation. A suitable pharmaceutical composition for inhalation may be an aerosol. An exemplary pharmaceutical composition for inhalation of a combination of the invention or any component thereof may include: an aerosol container with a capacity of 15-20 ml comprising the active ingredient (e.g., a platinum-based compound represented by structural formula I (e.g., satraplatin) and/or another chemotherapeutic agent), a lubricating agent, such as polysorbate 85 or oleic acid, dispersed in a propellant, such as freon, preferably in a combination of 1,2-dichlorotetrafluoroethane and difluorochloromethane. Preferably, the composition is in an appropriate aerosol container adapted for either intranasal or oral inhalation administration.

A property of satraplatin is that it is orally bioavailable. Although the scope of the present invention includes pharmaceutical compositions for parenteral administration to a subject, the present invention also includes pharmaceutical compositions comprising satraplatin that are suitable for oral administration. For example, in an embodiment of the invention, a pharmaceutical composition comprises satraplatin (e.g., a dosage form, such as a pill or tablet, comprising 10, 50 or 200 mg of satraplatin) and the excipients microcrystalline cellulose, sodium starch glycolate, lactose, and magnesium stearate (see e.g., Fokkema et al., J. Clin. Onco. 17(12): 3822-3827 (1999)).

### Therapeutic Methods and Administration

Compositions of the invention are useful, inter alia, for the prevention or treatment of cancer in a subject. Accordingly, the present invention comprises methods of using the compositions of the invention (e.g., Pt based compound in association with another chemotherapeutic agent) for the treatment or prevention of cancer.

The term “subject” or “patient” includes any organism, preferably a mammal (e.g., primate, dog, horse, rat, mouse, cat, rabbit) and most preferably a human.

Typical protocols for the therapeutic administration of such compositions are well known in the art. Pharmaceutical composition of the invention may be administered, for example, by any parenteral (e.g., subcutaneous injection, intramuscular injection, intravenous injection) or non-parenteral route (e.g., orally, nasally).

Pills and capsules of the invention can be administered orally. Injectable compositions can be administered with medical devices known in the art; for example, by injection with a hypodermic.

Injectable pharmaceutical compositions of the invention may also be administered with a needleless hypodermic injection device; such as the devices disclosed in U.S. Pat. Nos. 5,399,163; 5,383,851; 5,312,335; 5,064,413; 4,941,880; 4,790,824 or 4,596,556.

The daily dose of a chemotherapeutic agent of the present invention is, where possible, administered accordance with the Physicians' Desk Reference 2003 (Physicians' Desk Reference, 57th Ed); Medical Economics Company; ISBN: 1563634457; 57th edition (November 2002). The proper dosage can, however, be altered by a clinician to compensate for particular characteristics of the subject receiving the therapy depending, for example, on the potency of the compound administered, side-effects, age, weight, medical condition, overall health and response.

The term “therapeutically effective amount” or “therapeutically effective dosage” means that amount or dosage of a composition of the invention that will elicit a biological or medical response of a tissue, system, subject or host that is being sought by the administrator (such as a researcher, doctor or veterinarian) which includes any measurable alleviation of the signs, symptoms and/or clinical indicia of cancer (e.g., tumor growth) and/or the prevention, slowing or halting of progression or metastasis of cancer to any degree. For example, in one embodiment, a “therapeutically effective dosage” of a platinum-based compound represented by structural formula I (e.g., satraplatin) is about 10 mg/m²/day to about 80 mg/m²/day.

In an embodiment of the invention, a therapeutically effective dose of any compound characterized by structural formula II (e.g., temozolomide) is about 150 mg/m² orally. In an embodiment of the invention, a therapeutically effective dosage of a compound represented by structural formula (CXIII) (e.g., lonafarnib) is about 300 mg to about 400 mg per day orally. In another embodiment of the invention, a therapeutically effective FOLFOX regimen dosage is set forth by Machover et al., Ann. Oncol. 7:95-98 (1996); Lévi et al., Cancer 69:893-900 (1992); de Gramont et al., Eur. J. Cancer 34:619-626 (1998); de Gramont et al., Proc. Am. Soc. Clin. Oncol. 13:220 (1994); de Gramont et al. Eur. J. Cancer 33:214-219 (1997); André et al., Ann. Oncol. 9:1-3 (1998); André et al., J. Clin. Oncol. 17: 3560-3568 (1999); Tournigand et al., Proc. Am. Soc. Clin. Oncol. 17:274 (1998); or Maindrault-Goebel et al., Eur. J. Cancer 35:1338-1342 (1999).

The scope of the present invention includes methods for treating or preventing any cancer in a subject by administering a composition of the invention (e.g., Pt based compound in association with another chemotherapeutic agent) to the subject. The present invention includes embodiments wherein the cancer is prostate cancer, including hormone-refractory prostate cancer (HRPC), non-small cell lung cancer (NSCLC), small cell lung cancer (SCLC), head & neck cancer, esophageal cancer, gastric cancer, bladder cancer, breast cancer (e.g., metastatic breast cancer), ovarian cancer, colon cancer, rectal cancer, colorectal cancer, glioblastoma multiforme (GBM), glioma, anaplastic astrocytoma (AA) or metastatic melanoma.

In an embodiment of the invention comprises administering a platinum-based compound represented by structural formula I (e.g., satraplatin) in association with the FOLFOX regimen for the treatment or prevention of metastatic colorectal cancer. Another embodiment of the invention comprises administering a platinum-based compound represented by structural formula I (e.g., satraplatin) in association with lonafarnib for the treatment or prevention of ovarian cancer. A further embodiment of the invention comprises administering a platinum-based compound represented by structural formula I (e.g., satraplatin) in association with lonafarnib and an aromatase inhibitor (e.g., letrozole, anastrazole or exemestane) in the treatment or prevention of metastatic breast cancer. Yet another embodiment of the invention comprises administering a platinum-based compound represented by structural formula I (e.g., satraplatin) in association with temozolomide for the treatment or prevention of glioblastoma multiforme (GBM) or glioma or anaplastic astrocytoma (AA), metastatic melanoma or other cancers including lung cancer, ovarian cancer, lymphoma, head and neck cancer or prostate cancer. In an embodiment of the invention, a platinum-based compound represented by structural formula I (e.g., satraplatin) is administered in association with an anti-androgen including, but not limited to, bicalutamide, flutamide, nulutamide or megestrol acetate for the treatment or prevention of prostate cancer. In another embodiment of the invention, a platinum-based compound represented by structural formula I (e.g., satraplatin) is administered in association with a lutenizing hormone-releasing hormone agonist such as goserelin acetate, leuprolide acetate, triptorelin pamoate or the acetate salt of [D-Ser(Bu t ) 6,Azgly 10] (pyro-Glu-His-Trp-Ser-Tyr-D-Ser(Bu t)-Leu-Arg-Pro-Azgly-NH₂ acetate [C₅₉H₈₄N₁₈O₁₄·(C₂H₄O₂)x where x=1 to 2.4] for the treatment or prevention of prostate cancer. In a further embodiment of the invention, a platinum-based compound represented by structural formula I (e.g., satraplatin) is administered in association with herceptin for the treatment or prevention of metastatic breast. In yet another embodiment of the invention, an anti-insulin-like growth factor (anti-IGFR1; e.g., as discussed herein) antibody is administered in association with a platinum-based compound represented by structural formula I (e.g., satraplatin) for the treatment or prevention of metastatic breast or ovarian cancer. The present invention comprises administering a platinum-based compound represented by structural formula I (e.g., satraplatin) in association with an interferon or a pegylated interferon (e.g., as discussed herein) for the treatment or prevention of bladder cancer.

In another embodiment of the invention, platinum-based compound represented by structural formula I (e.g., satraplatin) is administered to a subject in association with an anti-cancer therapeutic procedure or method including surgical tumorectomy.

### Kits

Kits of the present invention include satraplatin in association with another chemotherapeutic agent, optionally combined with a pharmaceutically acceptable carrier, in a pharmaceutical formulation. In an embodiment of the invention, the pharmaceutical composition is a pharmaceutical dosage form such as a pill, a powder, an injectable liquid, a tablet, dispersible granules, a capsule, a cachet or a suppository. See for example, Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

In an embodiment of the invention, kits of the present invention also include information, for example in the form of a package insert, The form of the insert may take any form, such as paper or on electronic media such as a magnetically recorded medium (e.g., floppy disk) or a CD-ROM.

The package insert includes, in an embodiment, information concerning the pharmaceutical compositions and dosage forms in the kit. Generally, such information aids patients and physicians in using the enclosed pharmaceutical compositions and dosage forms effectively and safely. For example, the following information regarding a composition of the invention or any component thereof can be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

In an embodiment, satraplatin and the other chemotherapeutic agent are supplied in association with one another. For example, in an embodiment, the components are supplied as separate compositions or combined into a single composition. For example, satraplatin and lonafarnib can be supplied within a single, common pharmaceutical dosage form (e.g., pill or tablet) as in separate pharmaceutical dosage forms (e.g., two separate pills or tablets).

### EXAMPLES

The following examples are described with respect to combinations of a Pt compound with lonafarnib or temozolomide or an anti-IGFR antibody. As would be understood by those of ordinary skill in the art, the experimental protocols described could be used to demonstrate efficacious additivity or synergistic effects against any of a variety of cancers using any of the compositions or combinations described herein.

### Example 1

### Anti-Proliferative Effects of Combinations Comprising Pt Based Componds and Lonafarnib, Temozolomide or Anti-IGFR Antibody 15H12/19D12 LCF/HCA.

In this experiment, it is believed that combinations comprising lonafarnib and each of several Pt based compounds (infra) will be shown to synergistically inhibit proliferation of a malignant cell line. The experiments set forth below are performed essentially as Adjei et al. (Clin. Cancer Res. 7:1438-1445 (2001)) performed the anti-proliferative assays.

Proliferation Assay. The A549 cell line from American Type Culture Collection (Manassas, Va.) is grown in the following media containing 100 units/ml penicillin G, 100 mg/ml streptomycin, and 2 mM glutamine: A549 in RPMI 1640-5% (v/v) FBS (medium A).

After subconfluent monolayers are trypsinized, aliquots containing 500 A549 cells are plated in multiple 35-mm dishes containing 2 ml of medium A and incubated for 18-24 h at 37° C. to allow cells to attach. Graded concentrations of lonafarnib 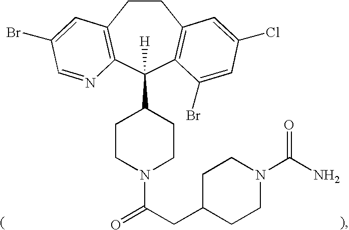
temozolomide 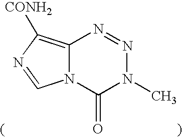
or anti-IGFR antibody (amino acids 20-128 of SEQ ID NO: 2 and amino acids 20-137 of SEQ ID NO: 4) alone, Pt based compounds alone 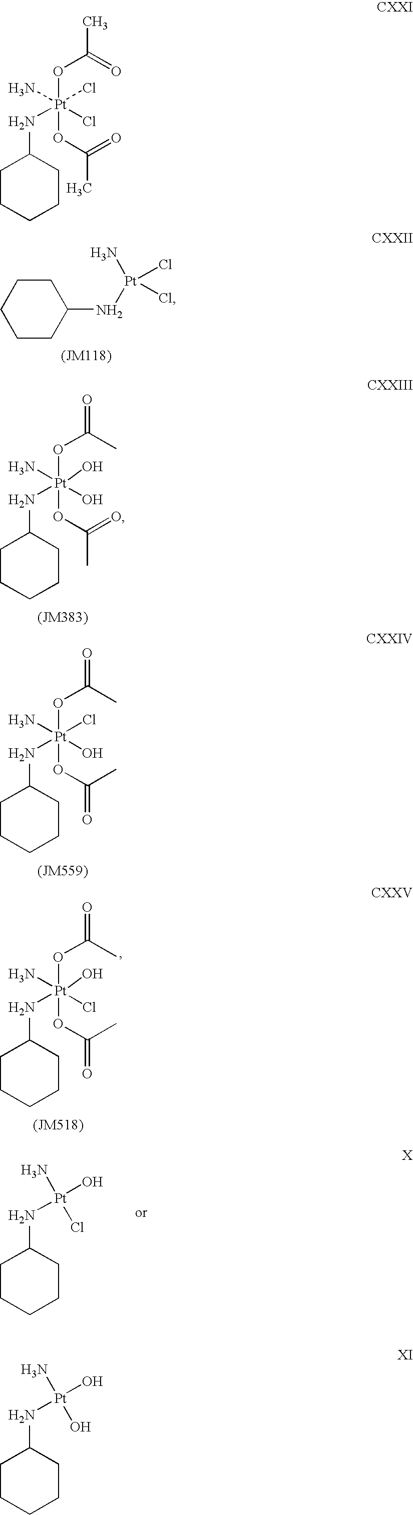
or combinations thereof or equivalent volumes of diluents are then added to triplicate plates, which are incubated for 7 days.

The concentration of lonafarnib and Pt based compound in each experiment is set forth below in Table 1.

TABLE 1
Concentration of Lonafarnib and Pt Based Compound to which
A549 Cells in Each Proliferation Assay are Exposed

| Lonafarnib, | |
|---|---|
| temozolomide or anti- | |
| IGFR antibody ((i) μM | |
| of lonafarnib or | |
| temozolomide, (ii) nM | Pt based compound |
| of antibody)^{¥} | (μM) |
| (i) 0, (ii) 0 | 0 |
| (i) 1, (ii) 5.12 × 10⁻⁵ | 0 |
| (i) 2, (ii) 2.56 × 10⁻⁴ | 0 |
| (i) 3, (ii) 1.28 × 10⁻³ | 0 |
| (i) 4, (ii) 3.2 × 10⁻² | 0 |
| (i) 5, (ii) 4 | 0 |
| (i) 6, (ii) 20 | 0 |
| (i) 0, (ii) 0 | 0.25 |
| (i) 0, (ii) 0 | 0.5 |
| (i) 0, (ii) 0 | 0.75 |
| (i) 0, (ii) 0 | 1.0 |
| (i) 0, (ii) 0 | 1.25 |
| (i) 0, (ii) 0 | 1.50 |
| (i) 1, (ii) 5.12 × 10⁻⁵ | 0.25 |
| (i) 2, (ii) 2.56 × 10⁻⁴ | 0.5 |
| (i) 3, (ii) 1.28 × 10⁻³ | 0.75 |
| (i) 4, (ii) 3.2 × 10⁻² | 1.0 |
| (i) 5, (ii) 4 | 1.25 |
| (i) 6, (ii) 20 | 1.50 |

^{¥}Quantity (i) refers to the micromolarity of lonafarnib or temozolomide used in combination with the Pt based compound; and quantity (ii) refers to the nanomolarity of antibody used in combination with the Pt based compound.

Each experiment is performed for each pair wise combination of Pt based compound set forth above and lonafarnib, temozolomide or anti-IGFR.

Resulting colonies are stained with Coomassie Blue and counted manually. Control plates typically contain 150-200 colonies.

Analysis of Combined Drug Effects. Dose-response curves are initially generated for each agent to estimate its IC50 for the cell line under study. In subsequent experiments, cells are treated with serial dilutions of each drug individually and with both drugs simultaneously. Cells are exposed continuously to the candidate drugs. Fractional survival (f) is calculated by dividing the number of colonies in drug-treated plates by the number of colonies in control plates. Data are analyzed by the method of Chou and Talalay (Adv. Enzyme Regul. 22:27-55 (1984)). In brief, log[(1/f)−1] is plotted against log(drug dose). From the resulting median effect curves, the X intercept (log IC50) and slope (m) are calculated for each drug and for the combination by the method of least squares. These parameters are then used to calculate doses of the individual drugs and the combination required to produce varying levels of cytotoxicity (f=5 0.95, 0.90, 0.85, . . . , 0.05) according to the equation:
Dose_{f}=Dose_{IC50}[(1−f)/f]^{1/m}

Because the two drugs are administered at a fixed ratio, the dose of the combination required to produce fractional survival f could be divided into the component doses (D)₁ and (D)₂ of drugs 1 and 2, respectively. For each level of cytotoxicity, a parameter called the Cl is calculated according to the equation:
Cl=(D)₁/(D_{f})₁+(D)₂/(D_{f})₂+α(D)₁(D)₂/(D_{f})₁(D_{f})₂

where (D)₁ and (D)₂ are concentrations of the combination required to produce survival f, (D_{f})₁ and (D_{f})₂ are the concentrations of the individual drugs required to produce f, and α=1 or 0 depending on whether the drugs are assumed to be mutually nonexclusive or mutually exclusive, respectively. In this method, synergy is indicated by Cl<1, additivity by Cl=1, and antagonism by Cl>1 (Chou et al., Adv. Enzyme Regul. 22:27-55 (1984)).

The Cl value for each experiment wherein lonafarnib, temozolomide or anti-IGFR is combined with a Pt based compound is expected to be less than 1 which indicates that the anti-proliferative effect of the combined substances is synergistic.

### Example 2

### In Vivo Tumor Growth Inhibition of Lonafarnib or Temozolomide in Mice

One hundred 7-8 week old nude mice are subcutaneously injected with 5×10⁶ A549 non-small cell lung cancer cells and divided into 10 groups. Groups 1 and 2 are not treated or treated with vehicle control, respectively. Groups 3 and 4 are treated with lonafarnib or temozolomide at 60 mpk and 30 mpk respectively. Groups 5 and 6 are treated with 3 mpk Pt based compound and 1.5 mpk Pt based compound, respectively. Groups 7 and 8 are treated with the combined dose levels set forth above.

A separate experiment is performed for each pairwise combination including temozolomide or lonafarnib and the Pt based compounds set forth above, in Example 1.

Tumor growth inhibition is expressed as the tumor volume observed in mice treated with a substance being tested as a percentage of tumor volume in mice treated with vehicle only.

It is believed that the percentage of tumor growth inhibition of each individual Pt based compound measured will be synergistically enhanced. Specifically, that the sum of the percentage of inhibition of a Pt based compound alone and that of lonafarnib or temozolomide alone will be less than the percentage of inhibition observed when both a Pt based compound and either lonafarnib or temozolomide are coadministered.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. A composition comprising:
(a) a Pt based compound represented by the structural formula: 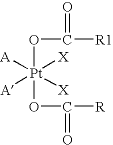
wherein A and A′ are independently 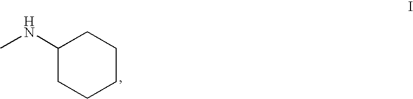
NH₃ or a C1-C10 cyclic, straight-chain or branched-chain alkyl amine; R and R1 are independently selected from the group consisting of hydrogen, C1-C10 alkyl, alkenyl, aryl, aralkyl, alkylamino and alkoxy; and X is selected from the group consisting of halogen, alkyl mono-carboxylate and alkyl di-carboxylate or a Pt based compound represented by the structural formula of 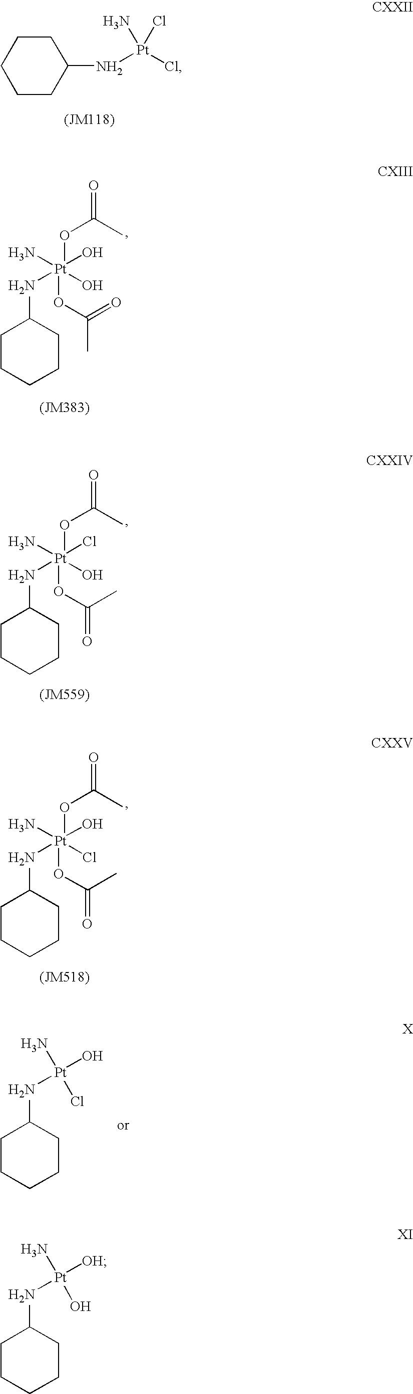
in association with
(b) another chemotherapeutic agent represented by the following structural formula: 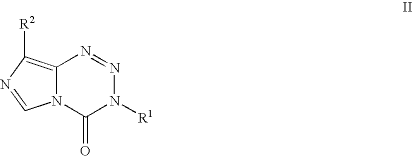
wherein R¹ represents a hydrogen atom, or a straight- or branched-chain alkyl, alkenyl or alkynyl group containing up to 6 carbon atoms, each such group being unsubstituted or substituted by from one to three substituents selected from halogen atoms, straight- or branched-chain alkoxy, alkylthio, alkylsullihinyl and alkylsulphonyl groups containing up to 4 carbon atoms, and optionally substituted phenyl groups, or wherein R¹ represents a cycloalkyl group; and R² represents a carbamoyl group optionally carries, on the nitrogen atom, one or two groups selected from straight- and branched-chain alkyl and alkenyl groups,each containing up to 4 carbon atoms, and cycloalkyl groups.

2. The composition of claim 1 wherein the Pt based compound is 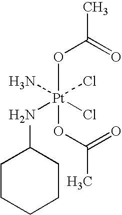
(satraplatin).

3. A pharmaceutical composition comprising the composition of claim 1 and a pharmaceutically acceptable carrier.

4. The pharmaceutical composition of claim 3 wherein the Pt based compound and the agent represented by structural formula II are formulated into a single oral dosage form.

5. The pharmaceutical composition of claim 4 wherein the oral dosage form is selected from the group consisting of a pill, a caplet, a tablet and a capsule.

6. A composition comprising:
(a) a Pt based compound represented by the structural formula: 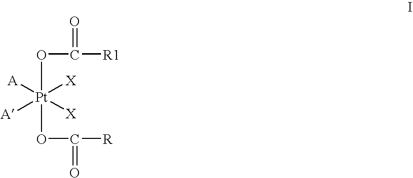
wherein A and A′ are NH₃ or a C1-C10 cyclic, straight-chain or branched-chain alkyl amine; R and R1 are selected from the group consisting of hydrogen, C1-C10 alkyl, alkenyl, aryl, aralkyl, alkylamino and alkoxy; and X is selected from the group consisting of halogen, alkyl mono- carboxylate and alkyl di-carboxylateor a Pt based compound represented by the structural formula 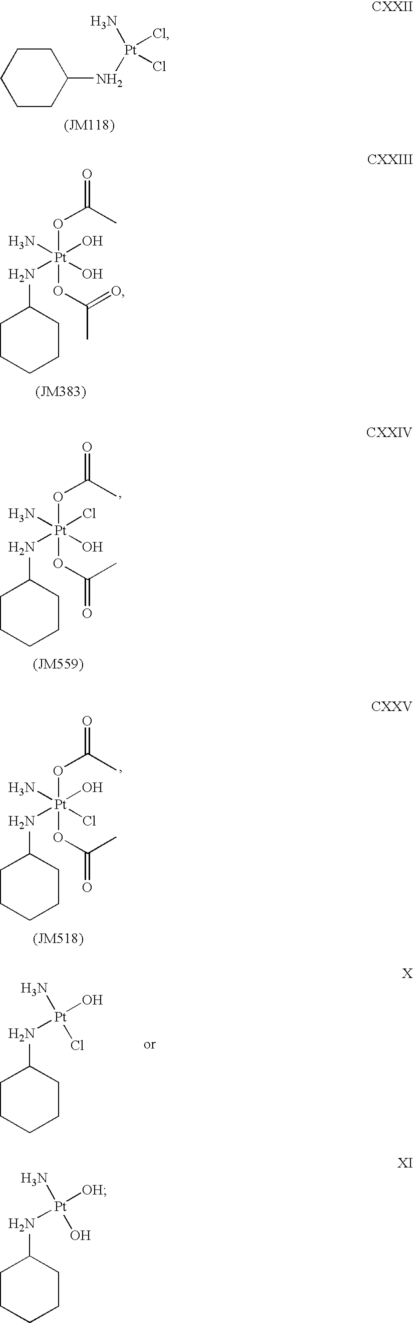
in association with
(b) another chemotherapeutic agent represented by the following structural formula: 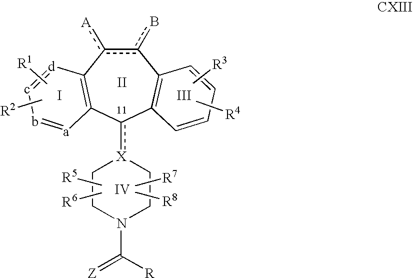
or a pharmaceutically acceptable salt or solvate thereof, wherein:
one of a, b, c and d represents N or NR9 wherein R9 is O—, —CH3 or —(CH2)nCO2H wherein n is 1 to 3, and the remaining a, b, c and d groups represent CR1 or CR2; or
each of a, b, c, and d are independently selected from CR1 or CR2;
each R1 and each R2 is independently selected from H, halo, —CF3, —OR10, —COR10, —SR10, —S(O)tR11 (wherein t is 0, 1 or 2), —SCN, —N(R10)2, —NO2, —OC(O)R10, —CO2R10, —OCO2R11, —CN, —NHC(O)R10, —NHSO2R10, —CONHR10, —CONHCH2CH2OH, —NR10COOR11, —SR11C(O)OR11, 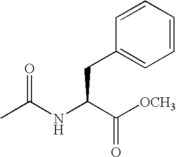
—SR11N(R75)2 (wherein each R75 is independently selected from H and —C(O)OR11), benzotriazol-1-yloxy, tetrazol-5-ylthio, or substituted tetrazol-5-ylthio, alkynyl, alkenyl or alkyl, said alkyl or alkenyl group optionally being substituted with halo, —OR10 or —CO2R10;
R3 and R4 are the same or different and each independently represents H, any of the substituents of R1 and R2, or R3 and R4 taken together represent a saturated or unsaturated C5-C7 fused ring to the benzene ring;
R5, R6, R7 and R8 each independently represents H, —CF3, —COR10, alkyl or aryl, said alkyl or aryl optionally being substituted with —OR10, —SR10, —S(O)tR11, —NR10COOR11, —N(R10)2, —NO2, —COR10, —OCOR10, —OCO2R11, —CO2R10, OPO3R10 or one of R5, R6, R7 and R8 can be taken in combination with R40 as defined below to represent —(CH2)r- wherein r is 1 to 4 which can be substituted with lower alkyl, lower alkoxy, —CF3 or aryl, or R5 is combined with R6 to represent ═O or ═S and/or R7 is combined with R8 to represent ═O or ═S;
R10 represents H, alkyl, aryl, or aralkyl;
R 11 represents alkyl or aryl;
X represents N, CH or C, which C may contain an optional double bond, represented by the dotted line, to carbon atom 11;
the dotted line between carbon atoms 5 and 6 represents an optional double bond, such that when a double bond is present, A and B independently represent —R10, halo, —OR11, —OCO2R11 or —OC(O)R10, and when no double bond is present between carbon atoms 5 and 6, A and B each independently represent H2, —(OR11)2; H and halo, dihalo, alkyl and H, (alkyl)2, —H and —OC(O)R10, H and —OR10, ═O, aryl and H, ═NOR10 or —O—(CH2)p-O— wherein p is 2, 3 or 4;
R represents R40, R42, R44, or R54, as defined below;
R40 represents H, aryl, alkyl, cycloalkyl, alkenyl, alkynyl or -D wherein -D represents 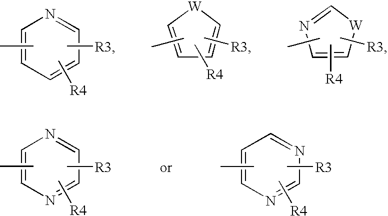
wherein R3 and R4 are as previously defined and W is O, S or NR10 wherein R10 is as defined above; said R40 cycloalkyl, alkenyl and alkynyl groups being optionally substituted with from 1-3 groups selected from halo, —CON(R10)2, aryl, —CO2R10, —OR12, —SR12, —N(R10)2, —N(R10)CO2R11, —COR12, —NO2 or D, wherein -D, R10 and R11 are as defined above and R12 represents R10, —(CH2)mOR10 or —(CH2)qCO2R10 wherein R10 is as previously defined, m is 1 to 4 and q is 0 to 4; said alkenyl and alkynyl R40 groups not containing —OH, —SH or
—N(R10)2 on a carbon containing a double or triple bond respectively; or
R40 represents phenyl substituted with a group selected from —SO2NH2, —NHSO2CH3, —SO2NHCH3, —SO2CH3, —SOCH3, —SCH3, or —NHSO2CF3, preferably, said group is located in the para position of the phenyl ring; or
R40 represents a group selected from 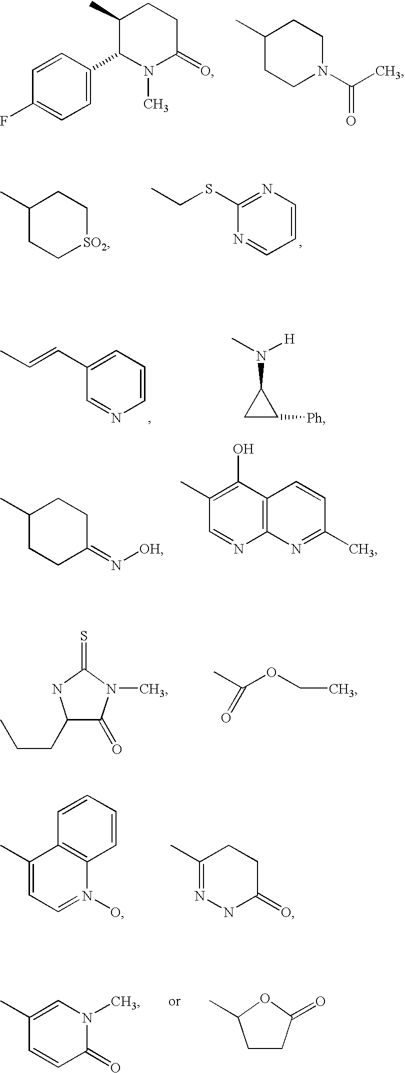
R42 represents 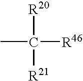
wherein R20, R21 and R46 are each independently selected from the group consisting of:
(1) H;
(2) —(CH2)qSC(O)CH3 wherein q is 1 to 3;
(3) —(CH2)qOSO2CH3 wherein q is 1 to 3;
(4) —OH;
(5) —CS(CH2)w(substituted phenyl) wherein w is 1 to 3 and the substitutents on said substituted phenyl group are the same substitutents as described below for said substituted phenyl;
(6) —NH2;
(7) —NHCBZ;
(8) —NHC(O)OR22 wherein R22 is an alkyl group having from 1 to 5 carbon atoms, or R22 represents phenyl substituted with 1 to 3 alkyl groups;
(9) alkyl;
(10) —(CH2)kphenyl wherein k is 1 to 6;
(11) phenyl;
(12) substituted phenyl wherein the substituents are selected from the group consisting of: halo, NO2, —OH, —OCH3, —NH2, —NHR22, —N(R22)2, alkyl, —O(CH2)tphenyl (wherein t is from 1 to 3), and —O(CH2)tsubstituted phenyl (wherein t is from 1 to 3);
(13) naphthyl;
(14) substituted naphthyl, wherein the substituents are as defined for substituted phenyl above;
(15) bridged polycyclic hydrocarbons having from 5 to 10 carbon atoms;
(16) cycloalkyl having from 5 to 7 carbon atoms;
(17) heteroaryl;
(18) hydroxyalkyl;
(19) substituted pyridyl or substituted pyridyl N-oxide wherein the substituents are selected from methylpyridyl, morpholinyl, imidazolyl, 1 piperidinyl, 1-(4-methylpiperazinyl), —S(O)tR11, or any of the substituents given above for said substituted phenyl, and said substitutents are bound to a ring carbon by replacement of the hydrogen bound to said carbon; 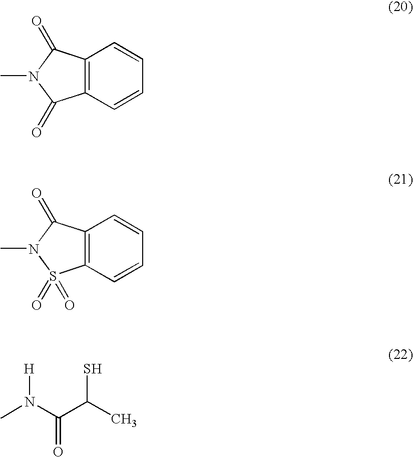
(23) —NHC(O)—(CH2)k-phenyl or —NH(O)—(CH2)k-substitued phenyl, wherein said k is as defined above;
(24) piperidine Ring V: 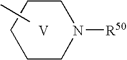
wherein R⁵⁰ represents H, alkyl, alkylcarbonyl, alkyloxycarbonyl, haloalkyl, or —C(O)NH(R¹⁰) wherein R¹⁰ is H or alkyl;
(25) —NHC(O)CH₂C₆H₅ or —NHC(O)CH₂-substituted-C₆H₅;
(26) —NHC(O)OC₆H₅; 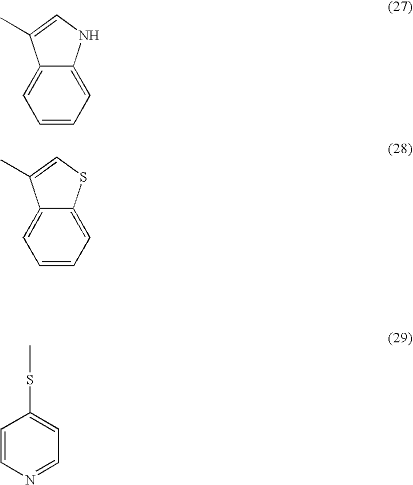
(30) —OC(O)-heteroaryl, for example 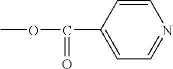
(31) —O-alkyl (e.g., —OCH3); and
(32) —CF3;
(33) —CN;
(34) a heterocycloalkyl group of the formula 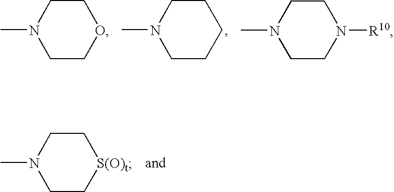
(35) a piperidinyl group of the formula 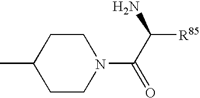
wherein R85 is H, alkyl, or alkyl substituted by —OH or —SCH3; or
R20 and R21 taken together form a ═O group and the remaining R46 is as defined above; or
Two of R20, R21 and R46 taken together form piperidine Ring V 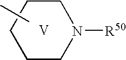
wherein R50 is as defined above;
with the proviso that R46, R20 and R21 are selected such that the carbon atom to which they are bound does not contain more than one heteroatom;
R44 represents 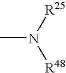
wherein R25 represents heteroaryl, N-methylpiperdinyl or aryl; and R48 represents H or alkyl;
R54 represents an N-oxide heterocyclic group of the formula (i), (ii), (iii) or (iv): 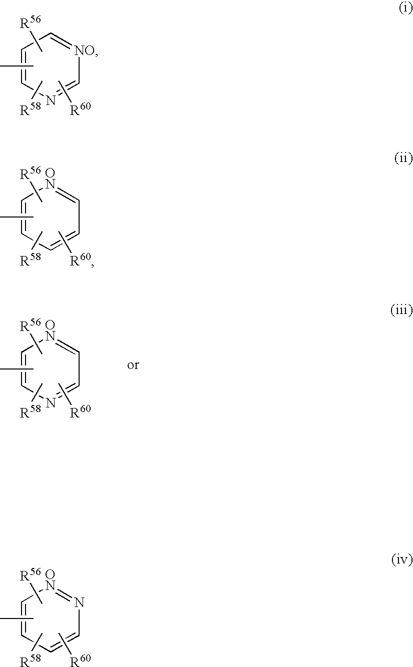
wherein R56, R58, and R60 are the same or different and each is independently selected from H, halo, —CF3, —OR10, —C(O)R10, —SR10,
—S(O)eR11 (wherein e is 1 or 2), —N(R10)2, —NO2, —CO2R10, —OCO2R11, —OCOR10, alkyl, aryl, alkenyl or alkynyl, which alkyl may be substituted with —OR10, —SR10 or —N(R10)2 and which alkenyl may be substituted with OR11 or SR11; or
R54 represents an N-oxide heterocyclic group of the formula (ia), (iia), (iiia) or (iva): 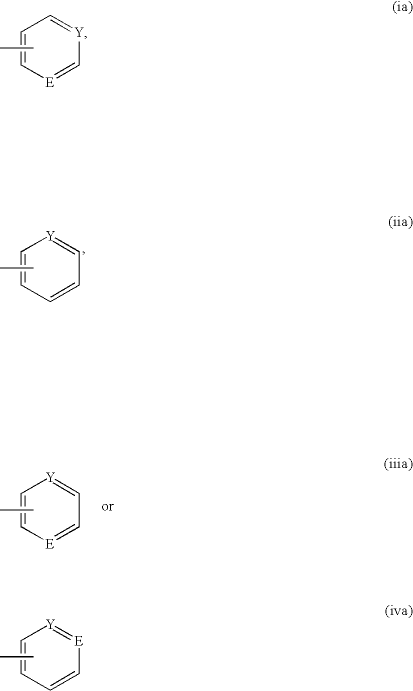
wherein Y represents N+—O— and E represents N; or
R54 represents an alkyl group substituted with one of said N-oxide heterocyclic groups (i), (ii), (iii), (iv), (ia), (iia), (iiia) or (iva);
Z represents O or S such that R can be taken in combination with R5, R6, R7 or R8 as defined above, or R represents R40, R42, R44 or R54.

7. The composition of claim 6 wherein the Pt based compound is 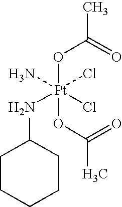
(satraplatin).

8. A pharmaceutical composition comprising the composition of claim 6 and a pharmaceutically acceptable carrier.

9. The pharmaceutical composition of claim 8 wherein the Pt based compound and the agent represented by structural formula CXIII are formulated into a singel oral dosage form.

10. The pharmaceutical composition of claim 9 wherein the oral dosage form is selected from the group consisting of a pill, a caplet, a tablet and a capsule.

11. A composition comprising:
(a) a Pt base d compound represented by the structural formula: 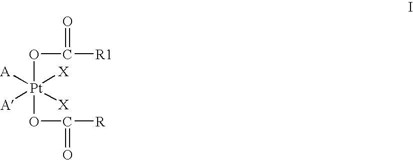
wherein A and A′ are NH₃ or a C1-C10 cyclic, straight-chain or branched-chain alkyl amine; R and R1 are selected from the group consisting of hydrogen, C1-C10 alkyl, alkenyl, aryl, aralkyl, alkylamino and alkoxy; and X is selected from the group consisting of halogen, alkyl mono-carboxylate and alkyl di-carboxylateor a Pt based compound represented by the structural formula 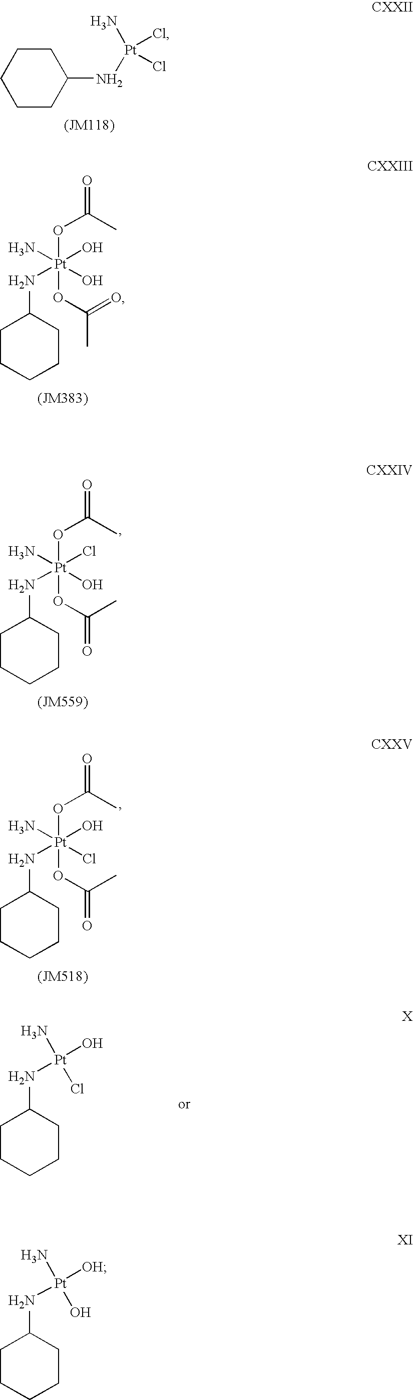
in association with
(b) another chemotherapeutic agent represented by a structural formula selected from the group consisting of: an isolated fully human monoclonal antibody that binds specifically to human IGFR1 comprising a light chain variable region comprising amino acids 20-128 or SEQ ID NO: 2 and a heavy chain variable region comprising amino acids 20-137 of SEQ ID NO: 4; 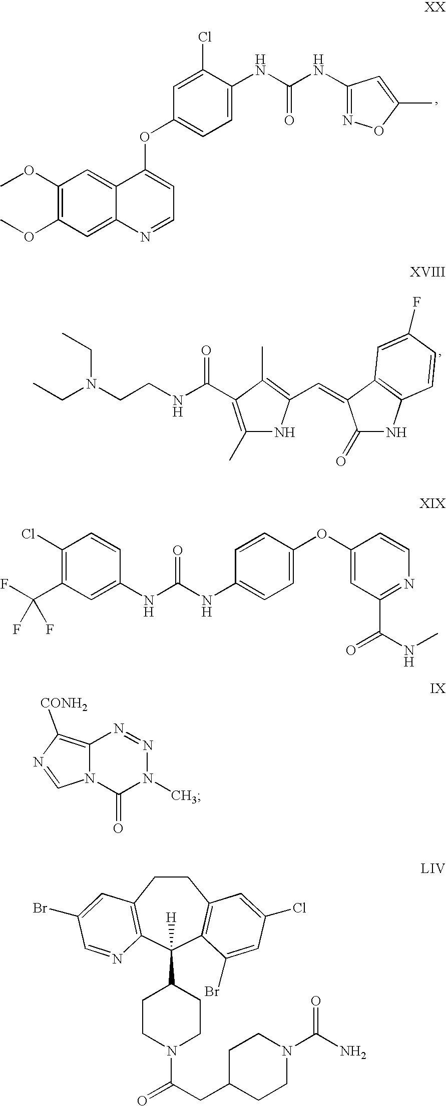
and a combination of (i) oxaliplatin 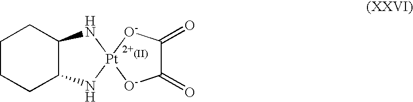
(ii) fluorouracil 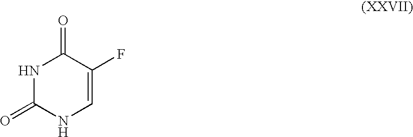
and (iii) folinic acid

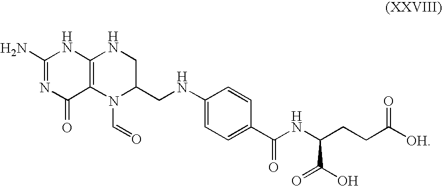

12. The composition of claim 11 wherein the Pt based compound is 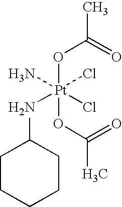
(satraplatin).

13. A pharmaceutical composition comprising the composition of claim 12 and a pharmaceutically acceptable carrier.

14. The pharmaceutical composition of claim 13 wherein the Pt based compound and an agent selected from the group consisting of 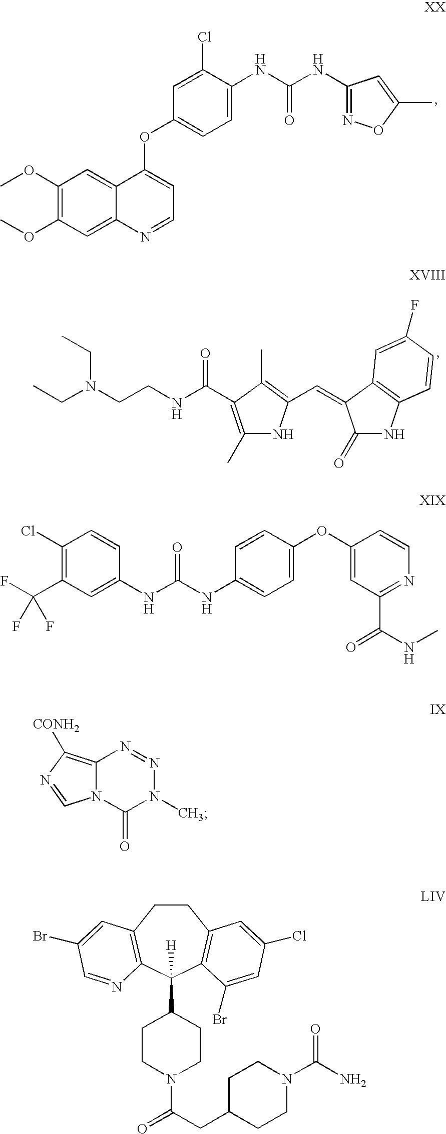
and a combination of (i) oxaliplatin 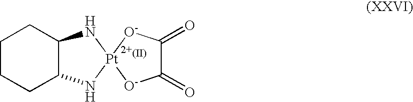
(ii) fluorouracil 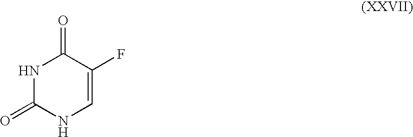
and (iii) folinic acid 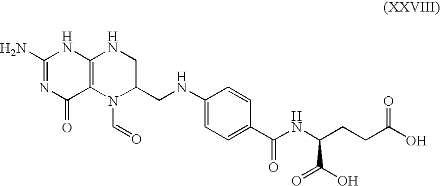
are formulated into a single oral dosage form.

15. The pharmaceutical composition of claim 14 wherein the oral dosage form is selected from the group consisting of a pill, a caplet, a tablet and a capsule.

16. A kit comprising in separate containers (a) a Pt based compound represented by the structural formula of: 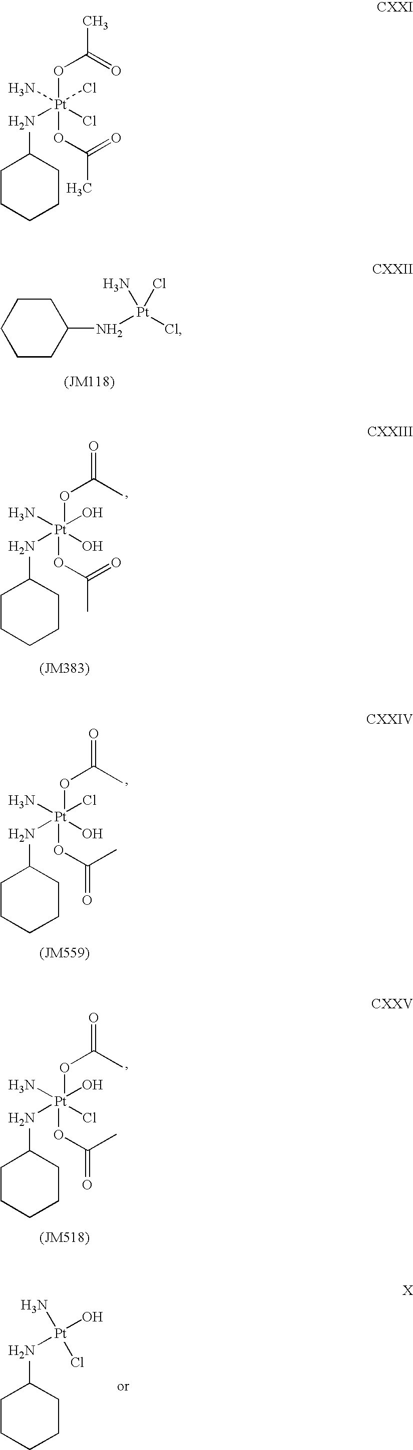 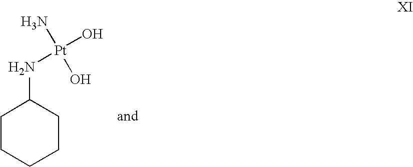
(b) a composition comprising a compound represented by a structural formula selected from the group consisting of:
an isolated fully human monoclonal antibody that binds specifically to human IGFR1 comprising a light chain variable region comprising amino acids 20-128 or SEQ ID NO: 2 and a heavy chain variable region comprising amino acids 20-137 of SEQ ID NO: 4; 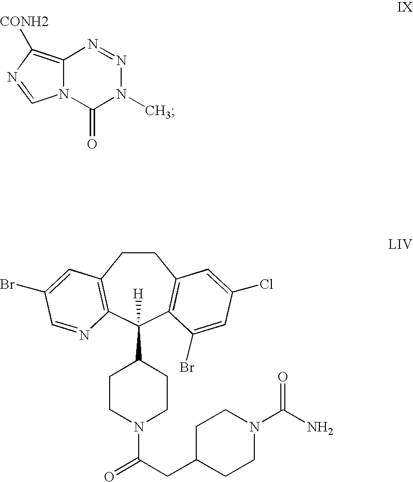
and a combination of (i) oxaliplatin 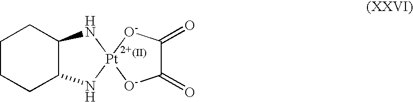
(ii) fluorouracil 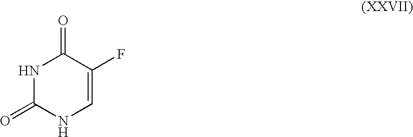
and (iii) folinic acid

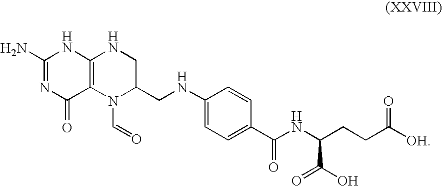

17. A method for treating or preventing cancer in a subject comprising administering, to the subject, a therapeutically effective amount of a composition according to any of claims 1, 6 or 11.

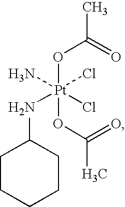

18. The method of claim 17 wherein the Pt based compound is

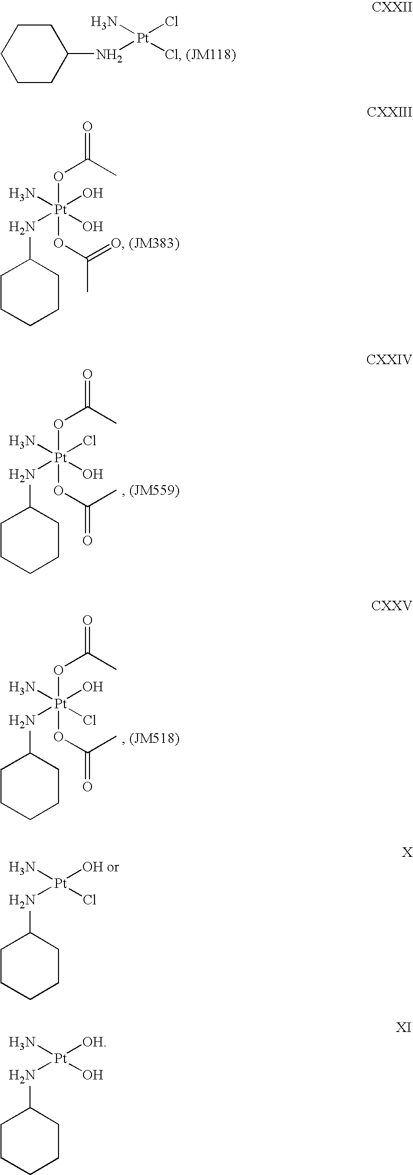

19. The method of claim 17 wherein the subject is also administered surgical tumorectomy.

20. The method of claim 17 wherein the cancer is selected from the group consisting of prostate cancer, hormone-refractory prostate cancer (HRPC), non-small cell lung cancer (NSCLC), small cell lung cancer (SCLC), head & neck cancer, esophageal cancer, gastric cancer, bladder cancer, breast cancer, ovarian cancer, rectal cancer, glioblastoma multiforme (GBM), glioma, anaplastic astrocytoma (AA), melanoma, colon cancer, colorectal cancer and metastatic melanoma.

21. The method of claim 17 wherein the subject is a human.

22. The method of claim 17 wherein satraplatin is administered to the subject orally.

23. The method of claim 17 wherein the other chemotherapeutic agent is administered to the subject parenterally.

24. The method of claim 17 wherein the other chemotherapeutic agent is administered to the subject non-parenterally.
